(19) United States
(12) Patent Application Publication
(10) Pub. No.: US 2021/0071261 A1
(43) Pub. Date: Mar. 11, 2021

(54) METHOD FOR DIAGNOSING CANCER BY MEANS OF BIOPSY CELL SAMPLE

(71) Applicant: LISEN IMPRINTING DIAGNOSTICS, INC., Dover, DE (US)

(72) Inventors: Tong CHENG, Wuxi (CN); Ning ZHOU, Wuxi (CN)

(73) Assignee: LISEN IMPRINTING DIAGNOSTICS, INC., Dover, DE (US)

(21) Appl. No.: 16/950,053

(22) Filed: Nov. 17, 2020

Related U.S. Application Data
(63) Continuation of application No. PCT/CN2018/087470, filed on May 18, 2018.

(51) Int. Cl. C12Q 1/6886

(52) U.S. Cl. CPC C12Q 1/6886; C12Q 2600/112; C12Q 2600/16

(57) ABSTRACT

The present application refers to a method for cancer diagnosis via biopsy samples. The method grades the expression status of imprinted genes by calculating the change of the expression of imprinted genes with loss of imprinting, the expression of imprinted genes with copy number variation, and the total expression of imprinted genes in tumor; wherein the imprinted genes are Z1 and/or Z16. Z1 is Gnas, and Z16 is Snrpn/Snurf. The method in the present application presents the expression of loss of imprinting in biopsy samples in a direct way for the first time, which can provide a quantitative model, to make a great contribution especially to the early diagnosis of transformation from tumor to cancer and screening of tumor.

### CROSS-REFERENCE TO RELATED APPLICATIONS

The present application is a continuation of International Application No. PCT/CN2018/087470, filed on May 18, 2018, which is hereby incorporated by reference in their entireties for all purposes.

### FIELD

The present application relates to the field of biotechnology, relates to the field of genetic diagnosis, specifically to a method for cancer diagnosis via biopsy cell samples.

### BACKGROUND

Cancer is a main disease that threatens human health. About 14 million cancer patients are newly diagnosed annually worldwide, and 8.2 million die annually from cancer, and the morbidity and mortality are still growing year by year. The development of cancer is gradual, so that the early-stage cancer is usually related to low malignancy, and a high five-year survival rate could be achieved in case of an immediate operative treatment. In contrast, the late-stage cancer is usually related to high malignancy and is more prone to metastasis, which hampers operative treatment and also leads to poor effectiveness in radiotherapy and chemotherapy. Therefore, the five-year survival rate is rather low in the late-stage cancer patients. Early diagnosis is a key step in cancer treatment, nevertheless, the early-stage cancer is often miss diagnosed due to its symptomless or atypical symptom. Imaging technologies such as ultrasound and CT are able to detect the early-stage cancer, while it is difficult to determine the benignity or malignancy without sampling and biopsy. The main methods currently used for biopsy sampling include fine needle aspiration, core needle aspiration biopsy, endoscope guided tissue biopsy, and the brush biopsy from bronchus, esophagus, oral cavity, and uterine cervix.

Fine needle aspiration (FNA) is a sampling method for detection of lump, which directly punctures into the larger lump palpable from the body surface through the hollow needle, or punctures for the deeper or smaller lump under the guidance of ultrasound or CT. FNA is widely used for the tumor sampling in thyroid, mammary gland, lymph node, parotid gland, pancreas, liver, lung, prostate, and ovary. In the traditional pathology, the benignity or malignancy of cells is determined by the cell size, shape, invasiveness, and their relationship with the surrounding tissue. Due to the small sample size obtained from FNA, the histological morphology is hard to be indicated, and in the meantime, the cell heteromorphism in the early-stage cancer is relatively low, which limits the accuracy of cytopathological diagnosis using FNA. In addition, FNA relies largely on the pathologist's experience, and certain diagnose may not achieved in some medium- and late-stage cancers.

The sampling method for core needle aspiration is similar with that of FNA, except for the coarser needle and bigger tissue stripe which can provide more histological information. However, the sample size is still small when compared with the whole tumor tissue, and some histological features, especially the invasive relationship between the cancer cells and the surrounding tissue is still difficult to determine. Therefore, limitation still exists in core needle aspiration.

Endoscope-guided tissue biopsy is a sampling method for suspicious lesion under the guidance of gastroscopy, colonoscopy, cystoscopy, hysteroscopy, or nasopharyngolarygnoscopy, which is usually used for the diagnosis of esophagus cancer, gastric cancer, colorectal cancer, bladder cancer, endometrial cancer, and nasopharyngeal cancer. Larger sample size can be obtained in tissue biopsy, but a definitive diagnosis is still hard in some early-stage cancer patients because of the low tissue heteromorphism.

Brush biopsy is often used for getting samples from bronchus, oral cavity, esophagus, and uterine cervix, and the small sample size is also difficult to show the histological morphology and resulted in low diagnostic accuracy. The urine exfoliated cell examination is a standard approach used in the detection of urinary system cancer, but the diagnosis is mainly given according to the cell morphology, and the diagnostic accuracy is rather low. The cystoscopy and biopsy are still necessary for a definitive diagnosis, in which patients may suffer much.

In order to avoid the limitation of traditional pathology in the early diagnosis of cancer, many biomarkers are developed for cancer diagnosis at the cell and molecule level, such as the BRAF for papillary thyroid carcinoma, the BRAC for breast cancer, the CEA for lung cancer, and the PSA for prostate cancer. However, some deficiencies still exist in these biomarkers, for example, BRAF can only be used for the detection of papillary thyroid carcinoma but not for the follicular thyroid carcinoma; the sensitivity of PSA is very high, but its specificity is rather low, giving rise to high false positive rate. So far, some tumor antigen detection and in situ hybridization technologies are applied in the urine, but the ideal sensitivity and specificity have not been achieved.

In the development of cancer, the changes in molecule level (epigenetics and genetics) is fairly ahead of these in cellular morphology and organizational structure, hence the molecular biology is more sensitive in detecting early stage cancer. Genomic imprinting is a kind of epigenetic regulation on the genes. For genomic imprinting, the allele from a specific parent is methylated and then silenced, so only the other allele is expressed. These genes are named imprinted genes. Loss of imprinting refers to a kind of epigenetic change in which the imprinted gene is demethylated and the silenced allele is reactivated for expression. Plenty of studies have revealed that this phenomenon (loss of imprinting) is common in various cancers, and is earlier than the morphological change in cell and tissue morphology. Meanwhile, in healthy cells, imprinting loss is very infrequent, which is in stark contrast with cancer cells. Therefore, the methylation status of the imprinted genes can be used as a pathological marker to analyze the abnormal condition of cells through certain molecular detecting technology. As shown in FIG. 1, the in situ detection for imprinted genes covers the shortages of the morphological pathology sensitively and effectively, wiping out the indeterminate zone of morphological pathology.

Based on these reasons, imprinted gene detection requires no histologic morphology and a small sample size, making it suitable for biopsy sample in cancer detection. The imprinted gene technology can be used for the detection of thyroid cancer, breast cancer, pancreatic cancer, lung cancer, liver cancer, colorectal cancer, bladder cancer, prostate cancer, gastric cancer, esophagus cancer, nasopharyngeal cancer, oral cancer, ovarian cancer, endometrial cancer, cervical cancer, central nervous system tumor, malignant parotid cancer, malignant lymphoma, or leukemia, and provide more accurate information for pre-diagnosis and final diagnosis, avoiding the problem of non-diagnosis due to insufficient cytological or histological heteromorphism. The imprinted gene detection can greatly increase the early diagnostic rate of cancer, and benefits for the patients by improving their life quality and prolonging their lifetime.

### SUMMARY

Considering the shortcomings of the present technologies, the present application provides a method for cancer diagnosis via biopsy samples. This method can observe the change of the imprinted genes at early stage cancer in a direct way, to determine the benignity or malignancy and the severity of cancer.

For the above propose, the present application uses the following technical solutions:

On one hand, the present application provides a method for cancer diagnosis via biopsy samples, comprising calculating the changes of the expressions of imprinted genes with loss of imprinting (LOI), the expressions of imprinted genes with copy number variation (CNV), and the total expressions (TE) of imprinted genes in a tumor, to grade the expressions of the imprinted genes;

wherein, the imprinted genes include one or a combination of imprinted gene Z1 and imprinted gene Z16, the imprinted gene Z1 is Gnas and the imprinted gene Z16 is Snrpn/Snurf.

In the present application, the inventors find that by calculating the LOI, CNV, and TE of the imprinted gene Z1, the sensitivity reaches 99.2% for thyroid cancer diagnosis, 99.8% for breast cancer, 90.0% for pancreatic cancer, 99.8% for lung cancer, and 89.0% for urinary system cancer.

In addition, the inventors find that by calculating the LOI, CNV, and TE of the imprinted gene Z16, the sensitivity reaches 99.8% for thyroid cancer diagnosis, 91.7% for breast cancer, 90.0% for pancreatic cancer, 90.0% for lung cancer, and 89.0% for urinary system cancer.

According to the present application, the inventors find that the sensitivity can be further increased by calculating the expression of Z1 and Z16 with loss of imprinting and the expression of Z1 and Z16 with copy number variation. With combination of Z1 and Z16, the sensitivity for diagnosis of thyroid cancer, breast cancer, pancreatic cancer, lung cancer, and urinary system cancer can be higher than 99.9%.

In some embodiments of the present application, the imprinted genes also include any one of Z2, Z3, Z4, Z5, Z6, Z7, Z8, Z9, Z10, Z11, Z12, Z13, Z14, or Z15, or the combination of at least two; wherein, Z2 is Igf2, Z3 is Peg10, Z4 is Igf2r, Z5 is Mest, Z6 is Plagl1, Z7 is Cdkn1c, Z8 is Dcn, Z9 is Dlk1, Z10 is Gatm, Z11 is Grb10, Z12 is Peg3, Z13 is Sgce, Z14 is Slc38a4, Z15 is Diras3.

In the present application, the inventors find that the addition of Z2, Z3, Z4, Z5, Z6, Z7, Z8, Z9, Z10, Z11, Z12, Z13, Z14, and Z15 on the basis of Z1 and Z16 helps to increase the accuracy of diagnosis. The inventors find that the sensitivities of imprinted genes Z2, Z3, Z4, Z5, Z6, Z7, Z8, Z9, Z10, Z11, Z12, Z13, Z14, and Z15 are different between cancers, which can be combined with other imprinted genes to determine the benignity and malignancy of cancer. Using the combination of the 16 imprinted genes Z1-Z16, the precise diagnosis of cancer can be achieved.

In some embodiments of the present application, the expression of an imprinted gene with loss of imprinting and the expression of an imprinted gene with copy number variation are calculated by the following formulas:

total expression=(b+c+d)/(a+b+c+d)×100%;

the expression of normal imprinted gene=b/(b+c+d)×100%;

the expression of an imprinted gene with loss of imprinting=c/(b+c+d)×100%;

the expression of an imprinted genes with copy number variation=d/(b+c+d)×100%;

wherein, “a” represents cell nuclei with no mark inside after performing hematoxylin staining on cells, which means the imprinted gene has no expression in the cell nuclei; “b” represents cell nuclei with one red/brown mark inside after performing hematoxylin staining on cells, which means the imprinted gene exists in the cell nuclei; “c” represents cell nuclei with two red/brown marks inside after performing hematoxylin staining on cells, which means the imprinted gene loses imprinting in the cell nuclei; and “d” represents cell nuclei with more than two red/brown marks inside after performing hematoxylin staining on cells, which means the imprinted gene has copy number variations in the cell nuclei.

In the present application, for an imprinted gene with loss of imprinting, after performing hematoxylin staining on cells, there are two red/brown marks in the cell nuclei. For an imprinted gene with copy number variation, after performing hematoxylin staining on cells, there are more than two red/brown markers in the cell nuclei. The copy number variation is caused by the abnormal gene duplication in cancer cells, resulting in the expression of this gene as a triploid or even higher polyploid.

In the present application, the mark after hematoxylin staining is, but not limited to, red or brown, and the marks after other staining method with other colors can also be used for calculating the expression of an imprinted gene, the expression of an imprinted gene with loss of imprinting and the expression of an imprinted gene with copy number variation.

In some embodiments of the present application, the expression of an imprinted gene with loss of imprinting, the expression of an imprinted gene with copy number variation, and the total expression of an imprinted gene are classified into 5 grades.

In some embodiments of the present application, the 5 grades are classified respectively according to the expression of an imprinted gene with loss of imprinting, the expression of an imprinted gene with copy number variation, and the total expression of an imprinted gene of imprinted genes Z1 and Z16.

In some embodiments of the present application, the expression of Z1 and Z16 with loss of imprinting, the expression of Z1 and Z16 with copy number variation, and the total expression of Z1 and Z16 are classified into 5 grades:

Grade 0: any one or the combination of at least two of: the expression of Z1 or Z16 with loss of imprinting is less than 15%, the expression of Z1 and Z16 with copy number variation in is less than 2%, or the total expression of Z1 and Z16 is less than 25%;

Grade I: any one or the combination of at least two of: the expression of Z1 or Z16 with loss of imprinting is 15-20%, the expression of Z1 and Z16 with copy number variation in is 2-4%, or the total expression of Z1 and Z16 is 25-30%;

Grade II: any one or the combination of at least two of: the expression of Z1 or Z16 with loss of imprinting is 20-25%, the expression of Z1 and Z16 with copy number variation in is 4-8%, or the total expression of Z1 and Z16 is 30-40%;

Grade III: any one or the combination of at least two of: the expression of Z1 or Z16 with loss of imprinting is 25-35%, the expression of Z1 and Z16 with copy number variation in is 8-12%, or the total expression of Z1 and Z16 is 40-50%;

Grade IV: any one or the combination of at least two of: the expression of Z1 or Z16 with loss of imprinting is more than 35%, the expression of Z1 and Z16 with copy number variation in is more than 12%, or the total expression of Z1 and Z16 is more than 50%.

In the present application, the expression of an imprinted gene with loss of imprinting, the expression of an imprinted gene with copy number variation, and the total expression of imprinted genes Z1 and Z16 are independent to each other.

In this application, the sensitivities of imprinted genes are different between cancers, and each index may fluctuate by 20% between cancers.

In some embodiments of the present application, the tumors include any one of thyroid tumor, breast tumor, pancreatic tumor, lung tumor, liver tumor, colorectal tumor, bladder tumor, prostate tumor, gastric tumor, esophagus tumor, nasopharyngeal tumor, oral tumor, ovarian tumor, endometrial tumor, cervical tumor, central nervous system tumor, parotid tumor, lymphoma, or leukemia, or the combination of at least two of them.

In some embodiments of the present application, for thyroid tumor, the expression of Z1 with loss of imprinting, the expression of Z1 with copy number variation, and the total expression of Z1 are classified into 5 grades:

Grade 0: any one or the combination of at least two of: the expression of Z1 with loss of imprinting is less than 15%, the expression of Zlwith copy number variation is less than 1.5%, or the total expression of Z1 is less than 40%;

Grade I: any one or the combination of at least two of: the expression of Z1 with loss of imprinting is 15-20%, the expression of Zlwith copy number variation is 1.5-4%, or the total expression of Z1 is 40-45%;

Grade II: any one or the combination of at least two of: the expression of Z1 with loss of imprinting is 20-30%, the expression of Zlwith copy number variation is 4-8%, or the total expression of Z1 is 45-60%;

Grade III: any one or the combination of at least two of: the expression of Z1 with loss of imprinting is 30-40%, the expression of Zlwith copy number variation is 8-15%, or the total expression of Z1 is 60-65%;

Grade IV: any one or the combination of at least two of: the expression of Z1 with loss of imprinting is more than 40%, the expression of Zlwith copy number variation is more than 15%, or the total expression of Z1 is more than 65%.

For thyroid tumor, the expression of Z16 with loss of imprinting, the expression of Z16 with copy number variation, and the total expression of Z16 are classified into 5 grades:

Grade 0: any one or the combination of at least two of: the expression of Z16 with loss of imprinting is less than 15%, the expression of Z16 with copy number variation is less than 1.5%, or the total expression of Z16 is less than 30%;

Grade I: any one or the combination of at least two of: the expression of Z16 with loss of imprinting is 15-20%, the expression of Z16 with copy number variation is 1.5-4%, or the total expression of Z16 is 30-35%;

Grade II: any one or the combination of at least two of: the expression of Z16 with loss of imprinting is 20-30%, the expression of Z16 with copy number variation is 4-8%, or the total expression of Z16 is 35-50%;

Grade III: any one or the combination of at least two of: the expression of Z16 with loss of imprinting is 30-40%, the expression of Z16 with copy number variation is 8-15%, or the total expression of Z16 is 50-55%;

Grade IV: any one or the combination of at least two of: the expression of Z16 with loss of imprinting is more than 40%, the expression of Z16 with copy number variation is more than 15%, or the total expression of Z16 is more than 55%.

In some embodiments of the present application, the inventor found that combining Z3, Z11, Z13 with Z1 and Z16 can increase the diagnostic sensitivity for thyroid tumor. For example, when a combination of Z1 and Z3 were detected, the diagnostic sensitivity can reach 99.9% or more, when a combination of Z1 and Z11 were detected, the diagnostic sensitivity can reach 99.9% or more, when a combination of Z1 and Z13 were detected, the diagnostic sensitivity can reach 99.9% or more, when a combination of Z11 and Z16 were detected, the diagnostic sensitivity can reach 99.9% or more, when a combination of Z13 and Z16 were detected, the diagnostic sensitivity can reach 99.9% or more.

For breast tumor, the expression of Z1 and Z16 with loss of imprinting, the expression of Z1 and Z16 with copy number variation, and the total expression of Z1 and Z16 are classified into 5 grades:

Grade 0: any one or the combination of at least two of: the expression of Z1 and Z16 with loss of imprinting is less than 15%, the expression of Z1 and Z16 with copy number variation is less than 1%, or the total expression of Z1 and Z16 is less than 25%;

Grade I: any one or the combination of at least two of: the expression of Z1 and Z16 with loss of imprinting is 15-20%, the expression of Z1 and Z16 with copy number variation is 1-3%, or the total expression of Z1 and Z16 is 25-30%;

Grade II: any one or the combination of at least two of: the expression of Z1 and Z16 with loss of imprinting is 20-25%, the expression of Z1 and Z16 with copy number variation is 3-7%, or the total expression of Z1 and Z16 is 30-40%;

Grade III: any one or the combination of at least two of: the expression of Z1 and Z16 with loss of imprinting is 25-30%, the expression of Z1 and Z16 with copy number variation is 7-10%, or the total expression of Z1 and Z16 is 40-50%;

Grade IV: any one or the combination of at least two of: the expression of Z1 and Z16 with loss of imprinting is more than 30%, the expression of Z1 and Z16 with copy number variation is more than 10%, or the total expression of Z1 and Z16 is more than 50%.

In the present application, the expression of an imprinted gene with loss of imprinting, the expression of an imprinted gene with copy number variation, and the total expression of imprinted genes Z1 and Z16 are independent to each other.

In some embodiments of the present application, the inventor found that combining Z8, Z10, and Z13 with Z1 and Z16 can increase the diagnostic sensitivity for breast tumor. For example, when a combination of Z1 and Z8 were detected, the diagnostic sensitivity can reach 99.9% or more, when a combination of Z1 and Z10 were detected, the diagnostic sensitivity can reach 99.9% or more, when a combination of Z1 and Z11 were detected, the diagnostic sensitivity can reach 99.9% or more, when a combination of Z1 and Z13 were detected, the diagnostic sensitivity can reach 99.9% or more, when a combination of Z1 and Z16 were detected, the diagnostic sensitivity can reach 99.9% or more.

For pancreatic tumor, the expression of Z1 and Z16 with loss of imprinting, the expression of Z1 and Z16 with copy number variation, and the total expression of Z1 and Z16 are classified into 5 grades:

Grade 0: any one or the combination of at least two of: the expression of Z1 and Z16 with loss of imprinting is less than 15%, the expression of Z1 and Z16 with copy number variation is less than 2%, or the total expression of Z1 and Z16 is less than 20%;

Grade I: any one or the combination of at least two of: the expression of Z1 and Z16 with loss of imprinting is 15-20%, the expression of Z1 and Z16 with copy number variation is 2-4%, or the total expression of Z1 and Z16 is 20-30%;

Grade II: any one or the combination of at least two of: the expression of Z1 and Z16 with loss of imprinting is 20-25%, the expression of Z1 and Z16 with copy number variation is 4-8%, or the total expression of Z1 and Z16 is 30-40%;

Grade III: any one or the combination of at least two of: the expression of Z1 and Z16 with loss of imprinting is 25-30%, the expression of Z1 and Z16 with copy number variation is 8-12%, or the total expression of Z1 and Z16 is 40-50%;

Grade IV: any one or the combination of at least two of: the expression of Z1 and Z16 with loss of imprinting is more than 30%, the expression of Z1 and Z16 with copy number variation is more than 12%, or the total expression of Z1 and Z16 is more than 50%.

In the present application, the expression of an imprinted gene with loss of imprinting, the expression of an imprinted gene with copy number variation, and the total expression of Z1 and Z16 are independent to each other.

In some embodiments of the present application, the inventor found that combining Z5, Z10, Z11 with Z1 and Z16 can increase the diagnostic sensitivity for pancreatic tumor. For example, when a combination of Z1 and Z5 were detected, the diagnostic sensitivity can reach 99.9% or more, when a combination of Z1 and Z10 were detected, the diagnostic sensitivity can reach 99.9% or more, when a combination of Z1 and Z16 were detected, the diagnostic sensitivity can reach 99.9% or more, when a combination of Z10 and Z16 were detected, the diagnostic sensitivity can reach 99.9% or more, when a combination of Z11 and Z16 were detected, the diagnostic sensitivity can reach 99.9% or more.

For lung tumor, the expression of Z1 with loss of imprinting, the expression of Z1 with copy number variation, and the total expression of Z1 are classified into 5 grades:

Grade 0: any one or the combination of at least two of: the expression of Z1 with loss of imprinting is less than 15%, the expression of Z1 with copy number variation is less than 2%, or the total expression of Z1 is less than 30%;

Grade I: any one or the combination of at least two of: the expression of Z1 with loss of imprinting is 15-20%, the expression of Z1 with copy number variation is 2-4%, or the total expression of Z1 is 30-40%;

Grade II: any one or the combination of at least two of: the expression of Z1 with loss of imprinting is 20-25%, the expression of Z1 with copy number variation is 4-8%, or the total expression of Z1 is 40-50%;

Grade III: any one or the combination of at least two of: the expression of Z1 with loss of imprinting is 25-30%, the expression of Z1 with copy number variation is 8-12%, or the total expression of Z1 is 50-60%;

Grade IV: any one or the combination of at least two of: the expression of Z1 with loss of imprinting is more than 30%, the expression of Z1 with copy number variation is more than 12%, or the total expression of Z1 is more than 60%.

For lung tumor, the expression of Z16 with loss of imprinting, the expression of Z16 with copy number variation, and the total expression of Z16 are classified into 5 grades:

Grade 0: any one or the combination of at least two of: the expression of Z16 with loss of imprinting is less than 10%, the expression of Z16 with copy number variation is less than 1%, or the total expression of Z16 is less than 25%;

Grade I: any one or the combination of at least two of: the expression of Z16 with loss of imprinting is 10-15%, the expression of Z16 with copy number variation is 1-2%, or the total expression of Z16 is 25-30%;

Grade II: any one or the combination of at least two of: the expression of Z16 with loss of imprinting is 15-20%, the expression of Z16 with copy number variation is 2-5%, or the total expression of Z16 is 30-40%;

Grade III: any one or the combination of at least two of: the expression of Z16 with loss of imprinting is 20-25%, the expression of Z16 with copy number variation is 5-8%, or the total expression of Z16 is 40-50%;

Grade IV: any one or the combination of at least two of: the expression of Z16 with loss of imprinting is more than 25%, the expression of Z16 with copy number variation is more than 8%, or the total expression of Z16 is more than 50%.

In some embodiments of the present application, the inventor found that combining Z3, Z8, Z11 with Z1 and Z16 can increase the diagnostic sensitivity for lung tumor. For example, when a combination of Z1 and Z3 were detected, the diagnostic sensitivity can reach 99.9% or more, when a combination of Z1 and Z11 were detected, the diagnostic sensitivity can reach 99.9% or more, when a combination of Z1 and Z16 were detected, the diagnostic sensitivity can reach 99.9% or more, when a combination of Z10 and Z16 were detected, the diagnostic sensitivity can reach 99.9% or more, when a combination of Z3 and Z16 were detected, the diagnostic sensitivity can reach 99.9% or more.

For urinary system tumor, the expression of Z1 and Z16 with loss of imprinting, the expression of Z1 and Z16 with copy number variation, and the total expression of Z1 and Z16 are classified into 5 grades:

Grade 0: any one or the combination of at least two of: the expression of Z1 and Z16 with loss of imprinting is less than 17%, the expression of Z1 and Z16 with copy number variation is less than 2%;

Grade I: any one or the combination of at least two of: the expression of Z1 and Z16 with loss of imprinting is 17-20%, the expression of Z1 and Z16 with copy number variation is 2-3%;

Grade II: any one or the combination of at least two of: the expression of Z1 and Z16 with loss of imprinting is 20-25%, the expression of Z1 and Z16 with copy number variation is 3-7%;

Grade III: any one or the combination of at least two of: the expression of Z1 and Z16 with loss of imprinting is 25-30%, the expression of Z1 and Z16 with copy number variation is 7-12%;

Grade IV: any one or the combination of at least two of: the expression of Z1 and Z16 with loss of imprinting is more than 30%, the expression of Z1 and Z16 with copy number variation is more than 12%.

In the present application, the expression of an imprinted gene with loss of imprinting, the expression of an imprinted gene with copy number variation, and the total expression of imprinted genes Z1 and Z16 are independent to each other.

In some embodiments of the present application, the inventor found that combining Z2, Z3, and Z4 with Z1 and Z16 can increase the diagnostic sensitivity for urinary system tumor. For example, when a combination of Z1 and Z5 were detected, the diagnostic sensitivity can reach 99.9% or more, when a combination of Z1 and Z10 were detected, the diagnostic sensitivity can reach 99.9% or more, when a combination of Z1 and Z16 were detected, the diagnostic sensitivity can reach 99.9% or more, when a combination of Z10 and Z16 were detected, the diagnostic sensitivity can reach 99.9% or more, when a combination of Z11 and Z16 were detected, the diagnostic sensitivity can reach 99.9% or more.

According to this application, the following steps are involved:

(1) obtaining a test sample;

(2) designing a probe specific for the sequence of the imprinted genes;

(3) performing in situ hybridization using the probe of step (2) to the test sample; and

(4) analyzing microscopic images and determining the expression status of the imprinted genes;

wherein, the analysis is performed by calculating the expressions of imprinted genes with loss of imprinting, the expressions of imprinted genes with copy number variation, and the total expressions of imprinted genes, and grading the expressions of imprinted genes with loss of imprinting, the expressions of imprinted genes with copy number variation, and the total expressions of imprinted genes to determine the benignity and malignancy of a tumor.

According to the present application, the test sample of step (1) is human tissues and/or cells.

In some embodiments of the present application, the test sample includes any one or the combination of at least two of aspiration biopsy cells, biopsy cells, exfoliated cells, blood samples, or brush biopsy samples.

In the present application, the sampling procedure of biopsy samples includes acquiring cells from human tumor tissues by puncture, clamp and brushing, or acquiring exfoliated cells from urine, sputum, faeces, plural effusion and ascites, and then immediately fixing the sample in 10% neutral buffered formaldehyde solution or through other fixation method, followed by mounting to glass slides. Because the biopsy is less invasive and the acquisition of urine, sputum and feces is non-invasive, the sampling process is easy, and the sample source can be located in contrast to the circulating blood, the biopsy has its special advantages as the experimental sample. The sampling procedure of blood includes acquiring the peripheral blood and lysing the red blood cell using a red blood cell lysis buffer, and then fixing the sample in 10% neutral buffered formaldehyde solution or through other fixation method, followed by mounting to glass slides.

In some embodiments of the present application, the aspiration biopsy cells include fine or core needle aspiration biopsy samples, among which any one or the combination of at least two of the fine or core needle aspiration biopsy samples from thyroid, mammary gland, pancreas, lung, liver, prostate, ovary, lymph node, and parotid are preferable.

In some embodiments of the present application, the biopsy cells include biopsy cells from any one or the combination of at least two of gastroscopy, colonoscopy, cystoscopy, hysteroscopy, or nasopharyngolarygnoscopy.

In some embodiments of the present application, the exfoliated cells include exfoliated cells from any one or the combination of at least two of urine, sputum, faeces, plural effusion, or ascites.

In some embodiments of the present application, the brush biopsy samples include the brushing samples from any one or the combination of at least two of bronchus, esophagus, oral cavity, or cervical.

In some embodiments of the present application, in situ hybridization is performed using the RNAscope in situ hybridization method which is specific for RNA, the RNAscope in situ hybridization is performed by using singleplex or multiplex color assay kit or singleplex or multiplex fluorescence assay kit, among which, the singleplex red/brown color assay kit or multiplex fluorescence assay kit are preferable.

In the present application, the inventor calculates LOI, CNV, and TE in at least 100 cells with imprinted gene expression, to determine the benignity or malignancy of tumors through the grade of LOI, CNV, and TE. The benignity or malignancy of a tumor is classified into benign tumor, cancer potential, early-stage cancer, medium-stage cancer, and late-stage cancer.

The tumor is determined as a benign tumor, if the expression of both Z1 and Z16 with loss of imprinting are lower than grade I; if the expression of only one of Z1 and Z16 with loss of imprinting is grade I; if the expression of only one of Z1 and Z16 with copy number variation is grade I;

The tumor is determined as cancer potential, if the expression of both Z1 and Z16 with loss of imprinting are grade I; if the expression of both Z1 and Z16 with copy number variation are grade I; if the expression of only one of Z1 and Z16 with loss of imprinting is grade I and the expression of only one of Z1 and Z16 with copy number variation is grade I; if the expression of only one of Z1 and Z16 with loss of imprinting is grade II; if the expression of only one of Z1 and Z16 with copy number variation is grade II;

The tumor is determined as an early-stage cancer; if the expression of both Z1 and Z16 with loss of imprinting are grade II, and/or the expression of both Z1 and Z16 with copy number variation are grade II; if the expression of only one of Z1 and Z16 with loss of imprinting is grade II and the expression of only one of Z1 and Z16 with copy number variation is grade II; if the expression of only one of Z1 and Z16 with loss of imprinting is grade III; if the expression of only one of Z1 and Z16 with copy number variation is grade III;

The tumor is determined as a medium-stage cancer; if the expression of both Z1 and Z16 with loss of imprinting are grade III; if the expression of both Z1 and Z16 with copy number variation are grade III; if the expression of only one of Z1 and Z16 with loss of imprinting is grade III and the expression of only one of Z1 and Z16 with copy number variation is grade III; if the expression of only one of Z1 or Z16 with loss of imprinting is grade IV; if the expression of only one of Z1 or Z16 with copy number variation is grade IV;

The tumor is determined as a late-stage cancer if the expression of both Z1 and Z16 with loss of imprinting are grade IV, and/or the expression of both Z1 and Z16 with copy number variation are grade IV.

Second, the present application provides the use of the method mentioned in the first part for cancer detection and/or treatment.

Compared with the existing technologies, the present application has the following beneficial effects:

(1) The method described in the present application showed the phenomenon of loss of imprinting in the aspiration biopsy samples of thyroid, mammary gland, prostate, and lymph node, the bronchial brushing cell sample of lung, exfoliated cell samples from urine, sputum, feces, and plural effusion, and biopsy cell samples from colonoscopy and cystoscopy. Through in situ labeling of imprinted genes, this method detects the change of the imprinted genes objectively, intuitively, early, and precisely. In addition, this method provides the quantitative model, and makes a significant contribution to the early diagnosis of thyroid cancer, breast cancer, pancreatic cancer, lung cancer, liver cancer, colorectal cancer, and urinary system cancer.

(2) The method described in the present application can provide quantitative detection results, to determine the type of tumor precisely, and grade the malignancy of tumor, which solves the problem that part of the cases cannot be clearly diagnosed by the traditional cytopathology and histopathology. Moreover, the method also breakthroughs the limitation of current tissue and cell morphological diagnosis, and provides assistance for the future targeted therapy.

(3) This method in the present application is able to distinguish the benignity and malignancy of the hurthle cell tumor (HCT) of thyroid at the molecule level, provides a solution to the difficulty in distinguishing the benignity and malignancy from tissue and cell morphology. It is also difficult for the morphological diagnosis to distinguish the follicular thyroid adenoma (FTA) from follicular thyroid cancer (FTC), which can be distinguished by this method on the cell level

(4) This method in the present application can be performed together with the immunohistochemical method, to diagnose the benignity or malignancy of pancreatic tumor accurately at early stage, and avoid the false negative and other side effects.

(5) For the tumors including breast cancer, lung cancer, and liver cancer that can metastasize through the lymphatic system, this method is not only able to determine the benignity or malignancy at early stage through the fine-needle aspiration biopsy, but also able to puncture the lymph nodes adjacent to the tumor to test the metastasis through lymphatic system.

(6) The method in the present application enables the clear distinction of pigmented nevus from skin melanoma and other skin diseases, especially for the differential diagnosis of early stage melanoma in situ, and resolving the difficulty in the morphological diagnosis.

(7) The method in the present application enables the early diagnosis of breast ductal adenocarcinoma in situ and distinguishing from the benign breast lesions.

(8) This method can accurately diagnose the early stage malignant potential and malignant of the polyp in colon, resolving the early diagnosis problem of colorectal cancer.

(9) This method can distinguish gastritis and gastric ulcer from early stage gastric cancer, resolving the early diagnosis problem of gastric cancer.

(10) This method is independent of the tissue morphology, therefore can also be used for the diagnosis of benignity or malignancy through the aspiration biopsy samples from prostate cancer, ovarian cancer, central nervous system tumor, malignant tumor in the parotid gland, and malignant lymphoma, the endoscopic biopsy samples from gastric cancer, esophagus cancer, nasopharynx cancer, and endometrial cancer, the brush biopsy samples from lung cancer, oral cancer, esophagus cancer, and cervical cancer, and the blood sample from leukemia.

(11) The method in the present application can diagnose the leukemia through a single drop of peripheral blood, which can avoid the large trauma caused by currently used bone marrow aspiration.

(12) The method proposed in the present application can screen the early urinary system cancer, lung cancer, and colorectal cancer through the exfoliated cells from urine, sputum, and faeces. The sample collection procedure is simple and with no need for professional operators, so the samples can be collected by the patient at home. This method is suitable for the large scale physical examination, post-operational and drug effect monitoring in urinary system cancer, especially the pre-operative and post-operative detection, and can provide solutions to the early detection of postoperative recurrence and immediate treatment.

(13) In the surgical operation, the benignity and malignancy of the paracancerous tissue is a key indicator to evaluate the patient's prognosis and survival. The method in the present application provides an important guidance for it.

(14) The technologies, methods, and targeted drugs which can lead to the silence, knockout or rearrangement of the disease-related imprinted genes discovered by the method in the present application can be used to guide the later treatment and pharmacy.

### BRIEF DESCRIPTION OF DRAWINGS

FIG. 1 shows the difference between the imprinted gene in situ detection method described in this application and the traditional morphology diagnostic method;

FIG. 2 shows the aspiration cell smear of the thyroid cancer with nucleus stained by hematoxylin, wherein, the letter “a” represents cell nuclei with no mark inside after performing hematoxylin staining on cells, which means the imprinted gene has no expression in the cell nuclei; the letter “b” represents cell nuclei with one red/brown mark inside after performing hematoxylin staining on cells, which means the imprinted gene exists in the cell nuclei; the letter “c” represents cell nuclei with two red/brown marks inside after performing hematoxylin staining on cells, which means the imprinted gene loses imprinting in the cell nuclei; the letter “d” represents cell nuclei with more than two red/brown marks inside after performing hematoxylin staining on a cell, which means the imprinted gene has a copy number variation in the cell nuclei;

FIGS. 3(a)-FIG. 3(e) show the expression status of 9 of the 16 imprinted genes mentioned in the present application in the aspiration cell smear of the thyroid cancer with different grades of malignancy, wherein: FIG. 3(a) indicates the expression status of the 9 genes in the aspiration cell smear of grade 0 thyroid tumor, FIG. 3(b) indicates the expression status of the 9 genes in the aspiration cell smear of grade I thyroid cancer, FIG. 3(c) indicates the expression status of the 9 genes in the aspiration cell smear of grade II thyroid cancer, FIG. 3(d) indicates the expression status of the 9 genes in the aspiration cell smear of grade III thyroid cancer, FIG. 3(e) indicates the expression status of the 9 genes in the aspiration cell smear of grade IV thyroid cancer;

FIGS. 4(a)-FIGS. 4(b) demonstrate the distribution range and grading standards of the loss of imprinting, copy number variation and the total expression of imprinted genes Z1 and Z16 in aspiration cell samples of 177 thyroid tumors, wherein: FIG. 4(a) shows the distribution range and grading standard of loss of imprinting, copy number variation and total expression of Z1 in aspiration cell samples of 177 thyroid tumors,; and FIG. 4(b) shows the distribution range and grading standard of loss of imprinting, copy number variation and the total expression of Z16 in aspiration cell samples of 177 thyroid tumors;

FIG. 5(a) indicates the intensity of the loss of imprinting of Z1, Z4, Z11, Z13, and Z16 in thyroid cancer, FIG. 5(b) indicates the intensity of the copy number variation of Z1, Z4, Z11, Z13, and Z16 in thyroid cancer, FIG. 5(c) indicates the intensity of the total expression of Z1, Z4, Z11, Z13, and Z16 in thyroid cancer, FIG. 5(d) indicates the intensity of the loss of imprinting of Z2, Z3, Z5, and Z6 in thyroid cancer, FIG. 5(e) indicates the intensity of the copy number variation of Z2, Z3, Z5, and Z6 in thyroid cancer, FIG. 5(f) indicates the intensity of the total expression of Z2, Z3, Z5, and Z6 in thyroid cancer, wherein, LOI stands for the expression of an imprinted gene with loss of imprinting, CNV stands for the expression of an imprinted gene with copy number variation, and TE stands for the total expression of an imprinted gene;

FIG. 6(a) indicates the intensity of the loss of imprinting, copy number variation, and total expression of the imprinted gene Z1, FIG. 6(b) indicates the intensity of the loss of imprinting, copy number variation, and total expression of the imprinted gene Z16, FIG. 6(c) indicates the intensity of the loss of imprinting, copy number variation, and total expression of the imprinted gene Z4, FIG. 6(d) indicates the intensity of the loss of imprinting, copy number variation, and total expression of the imprinted gene Z11, FIG. 6(e) indicates the intensity of the loss of imprinting, copy number variation, and total expression of the imprinted gene Z13, FIG. 6(f) indicates the intensity of the loss of imprinting, copy number variation, and total expression of the imprinted gene Z2, FIG. 6(g) indicates the intensity of the loss of imprinting, copy number variation, and total expression of the imprinted gene Z3, FIG. 6(h) indicates the intensity of the loss of imprinting, copy number variation, and total expression of the imprinted gene Z5, FIG. 6(i) indicates the intensity of the loss of imprinting, copy number variation, and total expression of the imprinted gene Z6, wherein, LOI is the expression level of an imprinted gene with loss of imprinting, CNV is the expression of an imprinted gene with copy number variation, and TE is the total expression of an imprinted gene;

FIG. 7(a)-FIG. 7(b) demonstrate the distribution range and grading standards of the loss of imprinting, copy number variation and the total expression of imprinted genes Z1 and Z16 in aspiration cell samples of 18 breast cancers, FIG. 7(a) shows the distribution range and grading standard of loss of imprinting, copy number variation and total expression of Z1 in aspiration cell samples of 18 breast cancers, FIG. 7(b) shows the distribution range and grading standard of loss of imprinting, copy number variation and total expression of Z16 in aspiration cell samples of 18 breast cancers;

FIG. 8(a) indicates the intensity of the loss of imprinting of Z1, Z8, Z10, Z11, Z13, and Z16 in breast cancer, FIG. 8(b) indicates the intensity of the copy number variation of Z1, Z8, Z10, Z11, Z13, and Z16 in breast cancers, FIG. 8(c) indicates the intensity of the total expression of Z1, Z8, Z10, Z11, Z13, and Z16 in breast cancers, FIG. 8(d) indicates the intensity of the loss of imprinting of Z3, Z4, Z5, Z6, and Z9 in breast cancer, FIG. 8(e) indicates the intensity of the copy number variation of Z3, Z4, Z5, Z6, and Z9 in breast cancer, FIG. 8(f) indicates the intensity of the total expression of Z3, Z4, Z5, Z6, and Z9 in breast cancer, Of which, LOI is the expression of an imprinted gene with loss of imprinting, CNV is the expression of an imprinted gene with copy number variation, and TE is the total expression of an imprinted gene;

FIG. 9(a) indicates the intensity of the loss of imprinting, copy number variation, and total expression of the imprinted gene Z1, FIG. 9(b) indicates the intensity of the loss of imprinting, copy number variation, and total expression of the imprinted gene Z16, FIG. 9(c) indicates the intensity of the loss of imprinting, copy number variation, and total expression of the imprinted gene Z8, FIG. 9(d) indicates the intensity of the loss of imprinting, copy number variation, and total expression of the imprinted gene Z10, FIG. 9(e) indicates the intensity of the loss of imprinting, copy number variation, and total expression of the imprinted gene Z11, FIG. 9(f) indicates the intensity of the loss of imprinting, copy number variation, and total expression of the imprinted gene Z13, FIG. 9(g) indicates the intensity of the loss of imprinting, copy number variation, and total expression of the imprinted gene Z3, FIG. 9(h) indicates the intensity of the loss of imprinting, copy number variation, and total expression of the imprinted gene Z4, FIG. 9(i) indicates the intensity of the loss of imprinting, copy number variation, and total expression of the imprinted gene Z5, FIG. 9(j) indicates the intensity of the loss of imprinting, copy number variation, and total expression of the imprinted gene Z6, FIG. 9(k) indicates the intensity of the loss of imprinting, copy number variation, and total expression of the imprinted gene Z9, wherein, LOI is the expression of an imprinted gene with loss of imprinting, CNV is the expression of an imprinted gene with copy number variation, and TE is the total expression of an imprinted gene;

FIGS. 10(a)-FIG. 10(b) demonstrate the distribution range and grading standards of the loss of imprinting, copy number variation and the total expression of imprinted genes Z1 and Z16 in aspiration cell samples of 21 pancreatic cancers, FIG. 10(a) shows the distribution range and grading standards of loss of imprinting, copy number variation and total expression of Z1 in aspiration cell samples of 21 pancreatic cancers, FIG. 10(b) shows the distribution range and grading standards of loss of imprinting, copy number variation and total expression of Z16 in aspiration cell samples of 21 pancreatic cancers;

FIG. 11(a) indicates the intensity of the loss of imprinting of Z1, Z3, Z10, Z11, and Z16 in pancreatic cancer, FIG. 11(b) indicates the intensity of the copy number variation of Z1, Z3, Z10, Z11, and Z16 in pancreatic cancer, FIG. 11(c) indicates the intensity of the total expression of Z1, Z3, Z10, Z11, and Z16 in pancreatic cancer, FIG. 11(d) indicates the intensity of the loss of imprinting of Z4, Z5, Z6, Z8, and Z13 in pancreatic cancer, FIG. 11(e) indicates the intensity of the copy number variation of Z4, Z5, Z6, Z8, and Z13 in pancreatic cancer, FIG. 11(f) indicates the intensity of the total expression of Z4, Z5, Z6, Z8, and Z13 in pancreatic cancer, FIG. 11(g) indicates the intensity of the loss of imprinting of Z2, Z9, Z12, Z14, and Z15 in pancreatic cancer, FIG. 11(h) indicates the intensity of the copy number variation of Z2, Z9, Z12, Z14, and Z15 in pancreatic cancer, FIG. 11(i) indicates the intensity of the total expression of Z2, Z9, Z12, Z14, and Z15 in pancreatic cancer, wherein, LOI means the expression level of genes with lost imprinting in the imprinted genes, CNV stands for the expression of gene with copy number variation in the imprinted genes, and TE stands for the total expression of imprinted genes;

FIG. 12(a) indicates the intensity of the loss of imprinting, copy number variation, and total expression of the imprinted gene Z1, FIG. 12(b) indicates the intensity of the loss of imprinting, copy number variation, and total expression of the imprinted gene Z16, FIG. 12(c) indicates the intensity of the loss of imprinting, copy number variation, and total expression of the imprinted gene Z3, FIG. 12(d) indicates the intensity of the loss of imprinting, copy number variation, and total expression of the imprinted gene Z10, FIG. 12(e) indicates the intensity of the loss of imprinting, copy number variation, and total expression of the imprinted gene Z11, FIG. 12(f) indicates the intensity of the loss of imprinting, copy number variation, and total expression of the imprinted gene Z4, FIG. 12(g) indicates the intensity of the loss of imprinting, copy number variation, and total expression of the imprinted gene Z5, FIG. 12(h) indicates the intensity of the loss of imprinting, copy number variation, and total expression of the imprinted gene Z6, FIG. 12(i) indicates the intensity of the loss of imprinting, copy number variation, and total expression of the imprinted gene Z8, FIG. 12(j) indicates the intensity of the loss of imprinting, copy number variation, and total expression of the imprinted gene Z13, FIG. 12(k) indicates the intensity of the loss of imprinting, copy number variation, and total expression of the imprinted gene Z2, FIG. 12(l) indicates the intensity of the loss of imprinting, copy number variation, and total expression of the imprinted gene Z9, FIG. 12(m) indicates the intensity of the loss of imprinting, copy number variation, and total expression of the imprinted gene Z12, FIG. 12(n) indicates the intensity of the loss of imprinting, copy number variation, and total expression of the imprinted gene Z14, FIG. 12(o) indicates the intensity of the loss of imprinting, copy number variation, and total expression of the imprinted gene Z15, wherein, LOI is the expression of an imprinted gene with loss of imprinting, CNV is the expression of an imprinted gene with copy number variation, and TE is the total expression of an imprinted gene;

FIG. 13(a)-FIG. 13(b) demonstrate the distribution range and grading standards of the loss of imprinting, the copy number variation and the total expression of Z1 and Z16 in 23 bronchial brush cell samples, FIG. 13(a) shows the distribution range and grading standards of the loss of imprinting, the copy number variation and the total expression of Z1 in 23 bronchial brush cell samples, FIG. 13(b) shows the distribution range and grading standards of the loss of imprinting, the copy number variation and the total expression of Z16 in 23 bronchial brush cell samples;

FIG. 14(a) indicates the intensity of the loss of imprinting of Z1, Z3, Z8, Z13, and Z16 in lung cancer, FIG. 14(b) indicates the intensity of the copy number variation of Z1, Z3, Z8, Z13, and Z16 in lung cancer,

FIG. 14(c) indicates the intensity of the total expression of Z1, Z3, Z8, Z13, and Z16 in lung cancer, FIG. 14(d) indicates the intensity of the loss of imprinting of Z4, Z10, and Z11 in lung cancer, FIG. 14(e) indicates the intensity of the copy number variation of Z4, Z10, and Z11 in lung cancer, FIG. 14(f) indicates the intensity of the total expression of Z4, Z10, and Z11 in lung cancer, Wherein, LOI is the expression of an imprinted gene with loss of imprinting, CNV is the expression of an imprinted gene with copy number variation, and TE stands for the total expression of an imprinted gene;

FIG. 15(a) indicates the intensity of the loss of imprinting, copy number variation, and total expression of the imprinted gene Z1, FIG. 15(b) indicates the intensity of the loss of imprinting, copy number variation, and total expression of the imprinted gene Z16, FIG. 15(c) indicates the intensity of the loss of imprinting, copy number variation, and total expression of the imprinted gene Z3, FIG. 15(d) indicates the intensity of the loss of imprinting, copy number variation, and total expression of the imprinted gene Z8, FIG. 15(e) indicates the intensity of the loss of imprinting, copy number variation, and total expression of the imprinted gene Z13, FIG. 15(f) indicates the intensity of the loss of imprinting, copy number variation, and total expression of the imprinted gene Z4, FIG. 15(g) indicates the intensity of the loss of imprinting, copy number variation, and total expression of the imprinted gene Z10, FIG. 15(h) indicates the intensity of the loss of imprinting, copy number variation, and total expression of the imprinted gene Z11, wherein, LOI is the expression of an imprinted gene with loss of imprinting, CNV is the expression of an imprinted gene with copy number variation, and TE is the total expression of an imprinted gene;

FIG. 16(a)-FIG. 16(b) demonstrate the distribution range and grading standards of the loss of imprinting, the copy number variation and the total expression of Z1 and Z16 used in 70 exfoliated cell samples from urine, FIG. 16(a) shows the distribution range and grading standards of the loss of imprinting, the copy number variation and the total expression of Z1 used in 70 exfoliated cell samples from urine, FIG. 16(b) shows the distribution range and grading standards of the loss of imprinting and the copy number variation of Z16 used in 70 exfoliated cell samples from urine;

FIG. 17(a) indicates the intensity of the loss of imprinting of Z1, Z2, Z3, Z10, and Z16 in urinary system cancer, FIG. 17(b) indicates the intensity of the copy number variation of Z1, Z2, Z3, Z10, and Z16 in urinary system cancer, FIG. 17(c) indicates the intensity of the loss of imprinting of Z4, Z5, Z6, Z8, Z9, and Z15 in urinary system cancer, FIG. 17(d) indicates the intensity of the copy number variation of Z4, Z5, Z6, Z8, Z9, and Z15 in urinary system cancer, wherein, LOI is the expression of an imprinted gene with loss of imprinting, and CNV is the expression of an imprinted gene with copy number variation;

FIG. 18(a) indicates the intensity of the loss of imprinting, copy number variation, and total expression of the imprinted gene Z1, FIG. 18(b) indicates the intensity of the loss of imprinting, copy number variation, and total expression of the imprinted gene Z16, FIG. 18(c) indicates the intensity of the loss of imprinting, copy number variation, and total expression of the imprinted gene Z2, FIG. 18(d) indicates the intensity of the loss of imprinting, copy number variation, and total expression of the imprinted gene Z3, FIG. 18(e) indicates the intensity of the loss of imprinting, copy number variation, and total expression of the imprinted gene Z10, FIG. 18(f) indicates the intensity of the loss of imprinting, copy number variation, and total expression of the imprinted gene Z4, FIG. 18(g) indicates the intensity of the loss of imprinting, copy number variation, and total expression of the imprinted gene Z5, FIG. 18(h) indicates the intensity of the loss of imprinting, copy number variation, and total expression of the imprinted gene Z6, FIG. 18(i) indicates the intensity of the loss of imprinting, copy number variation, and total expression of the imprinted gene Z8, FIG. 18(j) indicates the intensity of the loss of imprinting, copy number variation, and total expression of the imprinted gene Z9, FIG. 18(k) indicates the intensity of the loss of imprinting, copy number variation, and total expression of the imprinted gene Z15, wherein, LOI is the expression of an imprinted gene with loss of imprinting, and CNV is the expression of an imprinted gene with copy number variation;

FIG. 19(a) shows the expression status of the 16 imprinted genes in the colonoscopy biopsy samples from the benign colorectal tumor, FIG. 19(b) shows the expression status of the 16 imprinted genes in the colonoscopy biopsy samples from the malignant potential colorectal tumor, FIG. 19(c) shows the expression status of the 16 imprinted genes in the colonoscopy biopsy samples from the colorectal cancer;

FIG. 20(a) shows the expression status of the 16 imprinted genes in the cystoscopy biopsy samples from the benign bladder tumor, FIG. 20(b) shows the expression status of the 16 imprinted genes in the cystoscopy biopsy samples from the malignant potential bladder tumor, FIG. 20(c) shows the expression status of the 16 imprinted genes in the cystoscopy biopsy samples from the bladder cancer;

FIG. 21(a) shows the expression status of the 16 imprinted genes in the sputum cell samples from the benign lung tumor, FIG. 21(b) shows the expression status of the 16 imprinted genes in the sputum cell samples from the lung cancer;

FIG. 22(a) shows the expression status of the 16 imprinted genes in the aspiration cell smear from the benign liver tumor, FIG. 22(b) shows the expression status of the 16 imprinted genes in the aspiration cell smear from the liver cancer;

FIG. 23(a) shows the expression status of the 16 imprinted genes in the aspiration cell smear from the benign prostate tumor, FIG. 23(b) shows the expression status of the 16 imprinted genes in the aspiration cell smear from the prostate cancer;

FIG. 24(a) shows the expression status of the 16 imprinted genes in the aspiration cell smear from the benign breast tumor without metastasis, FIG. 24(b) shows the expression status of the 16 imprinted genes in the aspiration cell smear from the lymph node adjacent to the metastatic breast cancer;

FIG. 25(a) shows the expression status of the 16 imprinted genes in the plural effusion cell samples from the benign lung tumor, FIG. 25(b) shows the expression status of the 16 imprinted genes in the plural effusion cell samples from the lung cancer;

FIG. 26(a) shows the expression status of the 16 imprinted genes in the feces exfoliated cell samples from the benign colorectal tumor, FIG. 26(b) shows the expression status of the 16 imprinted genes in the feces exfoliated cell samples from the colorectal cancer;

FIG. 27(a) shows the expression status of the 16 imprinted genes in the blood samples from the healthy, FIG. 27(b) shows the expression status of the 16 imprinted genes in the blood samples from the leukemia patient.

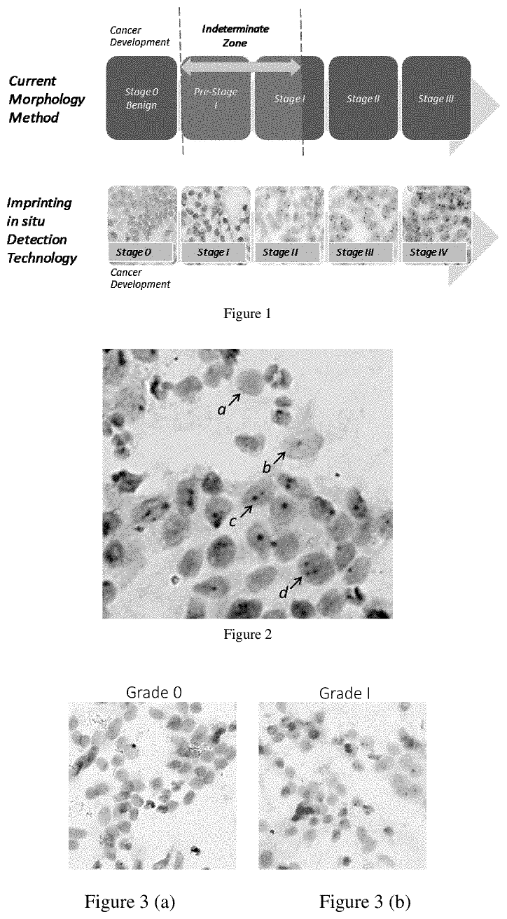

### SPECIFIC IMPLEMENTATION MODALITIES

To further elaborate the technological methods taken in the present application and their effects, the technological scheme in the present application is further explained below with reference to the attached figures and embodiments, but the present application is not limited to the scope of the embodiments.

### EXAMPLE 1

### Imprinted Gene Analysis in the Aspiration Cell Samples

The imprinted gene detection method includes the following steps:

(1) The aspiration cell sample was obtained using the biopsy needle, and then fixed in 10% neutral buffered formalin solution to protect the RNA from degradation. 24 h later, the fixed sample was proceeded for the cell smear slide preparation.

(2) The sample was pretreated according to the RNAScope sample pretreatment protocol to block the endogenous peroxidase activity, increase the permeability and expose the RNA molecules.

(3) Probe design: specific primers were designed according to the sequence of the imprinted genes.

The probes were designed according to the imprinted genes Z1 (Gnas), Z2 (Igf2), Z3 (Peg10), Z4 (Igf2r), Z5 (Mest), Z6 (Plagl1), Z7 (Cdkn1c), Z8 (Dcn), Z9 (Dlk1), Z10 (Gatm), Z11 (Grb10), Z12 (Peg3), Z13 (Sgce), Z14 (S1c38a4), Z15 (Diras3), and Z16 (Snrpn/Snurf). Specifically, a sequence within an intron of each gene was selected for probe design. Specific probes were design by Advanced Cell Diagnostics.

(4) RNAScope in situ hybridization was performed on the samples using the probes of step (3) according to the protocol of the kit.

(5) After signal amplification and hematoxylin staining, the expression of the imprinted genes was analyzed through microscopic imaging.

In the model of the present application, the formulas for calculating the expression of an imprinted gene, the expression of an imprinted gene with loss of imprinting and the expression of an imprinted gene with copy number variation are as follows:

Total expression of an imprinted gene=(b+c+d)/(a+b+c+d)×100%;

Expression of a normal imprinted gene=b/(b+c+d)×100%;

Expression of an imprinted gene with loss of imprinting=c/(b+c+d)×100%;

Expression of an imprinted gene with copy number variation=d/(b+c+d)×100%;

wherein, a, b, c, and d are illustrated in the FIG. 2, “a” represents cell nuclei with one red/brown mark inside after performing hematoxylin staining on cells, which means the imprinted gene has no expression in the cell nuclei; “b” represents cell nuclei with one red/brown mark inside after performing hematoxylin staining on cells, which means the imprinted gene exists in the cell nuclei; “c” represents cell nuclei with one red/brown mark inside after performing hematoxylin staining on cells, which means the imprinted gene loses imprinting in the cell nuclei; and “d” represents cell nuclei with one red/brown mark inside after performing hematoxylin staining on cells, which means the imprinted gene has copy number variation in the cell nuclei.

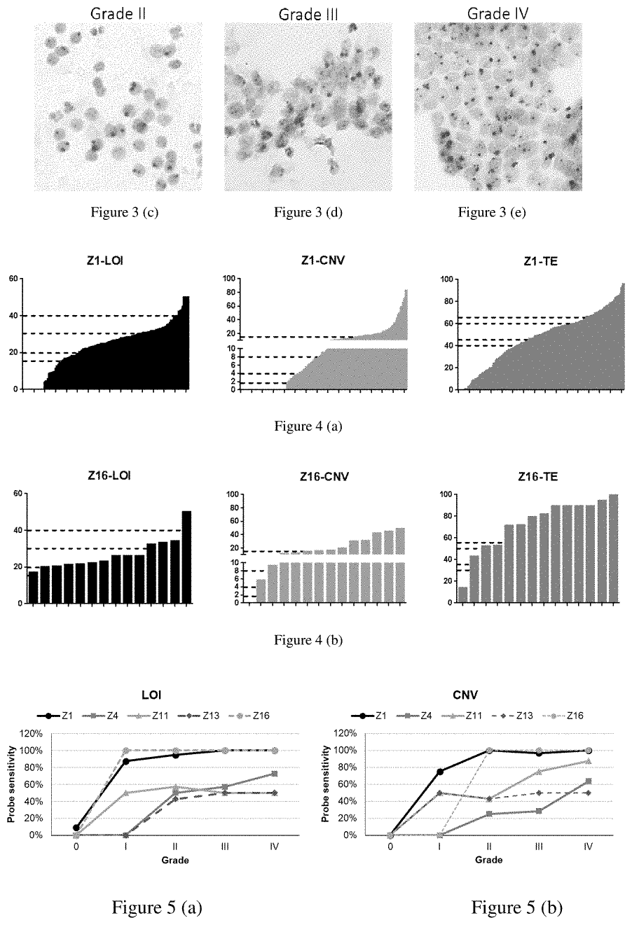

Take the thyroid cancer as an example, the detection results are shown in FIGS. 3(a)-3(e). As shown in FIGS. 3(a)-3(e), from grade 0 to grade IV, the proportion of the cells having imprinted gene with loss of imprinting (there are two marks in one nucleus) and cells having imprinted gene with copy number variation (there are three or more marks in one nucleus) increase with the stage of malignant tumor.

### EXAMPLE 2

### Imprinted Gene Analysis in 177 Aspiration Biopsy Cell Samples from Thyroid

177 thyroid aspiration biopsy cell samples were obtained by needle aspiration. Other methods were the same as in Example 1. The results are shown in FIGS. 4(a)-4 (b), FIGS. 5(a)-5(f), and FIGS. 6(a)-6(i).

As shown in FIG. 4(a), for the imprinted gene Z1, grade 0 is defined as when the expression of the imprinted gene with loss of imprinting is less than 15%, and/or the expression of imprinted gene with copy number variation is less than 1.5%, and/or the total expression of imprinted gene is less than 40%. Grade I is defined as when the expression of the imprinted gene with loss of imprinting is between 15-20%, and/or the expression of imprinted gene with copy number variation is between 1.5-4%, and/or the total expression of imprinted gene is between 40-45%. Grade II is defined as when the expression of the imprinted gene with loss of imprinting is between 20-30%, and/or the expression of imprinted gene with copy number variation is between 4-8%, and/or the total expression of imprinted gene is between 45-60%. Grade III is defined as when the expression of the imprinted gene with loss of imprinting is between 30-40%, and/or the expression of imprinted gene with copy number variation is between 8-15%, and/or the total expression of imprinted gene is between 60-65%. Grade IV is defined as when the expression of the imprinted gene with loss of imprinting is more than 40%, and/or the expression of imprinted gene with copy number variation is more than 15%, and/or the total expression of imprinted gene is more than 65%.

As shown in FIG. 4(b), for the imprinted gene Z16, grade 0 is defined as when the expression of imprinted gene with loss of imprinting is less than 15%, and/or the expression of imprinted gene with copy number variation is less than 1.5%, and/or the total expression of imprinted gene is less than 30%. Grade I is defined as when the expression of imprinted gene with loss of imprinting is between 15-20%, and/or the expression of imprinted gene with copy number variation is between 1.5-4%, and/or the total expression of imprinted gene is between 30-35%. Grade II is defined as when the expression of imprinted gene with loss of imprinting is between 20-30%, and/or the expression of imprinted gene with copy number variation is between 4-8%, and/or the total expression of imprinted gene is between 35-50%. Grade III is defined as when the expression of imprinted gene with loss of imprinting is between 30-40%, and/or the expression of imprinted gene with copy number variation is between 8-15%, and/or the total expression of imprinted gene is between 50-55%. Grade IV is defined as when the expression of imprinted gene with loss of imprinting is more than 40%, and/or the expression of imprinted gene with copy number variation is more than 15%, and/or the total expression of imprinted gene is more than 55%.

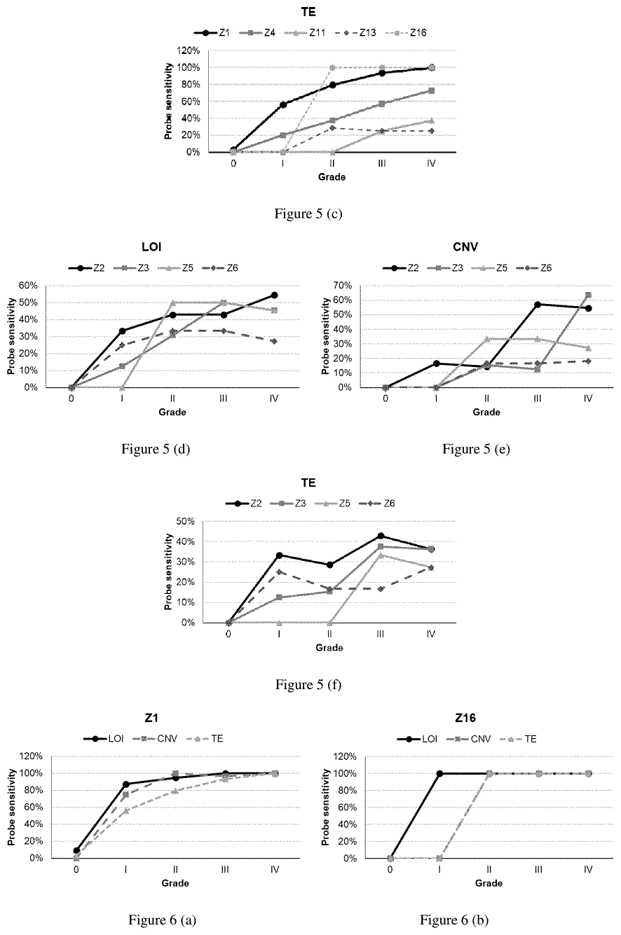

From FIGS. 5(a)-5(f), it can be observed that the sensitivity for thyroid cancer and the intensity and status of loss of imprinting in thyroid cancer is different between imprinted genes Z1, Z2, Z3, Z4, Z5, Z6, Z7, Z8, Z9, Z10, Z11, Z12, Z13, Z14, Z15, and Z16. Among them, Z1, Z16, Z4, Z11, and Z13 show higher sensitivities.

From the comprehensive analysis of these 177 aspiration biopsy cell samples from thyroid, it can be concluded that:

The thyroid tumor is determined as a benign tumor, if the expression of both Z1 and Z16 with loss of imprinting are lower than grade I; if the expression of only one of Z1 and Z16 with loss of imprinting is grade I; if the expression of only one of Z1 and Z16 with copy number variation is grade I;

The thyroid tumor is determined as thyroid cancer potential, if the expression of both Z1 and Z16 with loss of imprinting are grade I; if the expression of both Z1 and Z16 with copy number variation are grade I; if the expression of only one of Z1 and Z16 with loss of imprinting is grade I and the expression of only one of Z1 and Z16 with copy number variation is grade I; if the expression of only one of Z1 and Z16 with loss of imprinting is grade II; if the expression of only one of Z1 and Z16 with copy number variation is grade II;

The thyroid tumor is determined as an early-stage thyroid cancer; if the expression of both Z1 and Z16 with loss of imprinting are grade II, and/or the expression of both Z1 and Z16 with copy number variation are grade II; if the expression of only one of Z1 and Z16 with loss of imprinting is grade II and the expression of only one of Z1 and Z16 with copy number variation is grade II; if the expression of only one of Z1 and Z16 with loss of imprinting is grade III; if the expression of only one of Z1 and Z16 with copy number variation is grade III;

The thyroid tumor is determined as a medium-stage thyroid cancer; if the expression of both Z1 and Z16 with loss of imprinting are grade III; if the expression of both Z1 and Z16 with copy number variation are grade III; if the expression of only one of Z1 and Z16 with loss of imprinting is grade III and the expression of only one of Z1 and Z16 with copy number variation is grade III; if the expression of only one of Z1 or Z16 with loss of imprinting is grade IV; if the expression of only one of Z1 or Z16 with copy number variation is grade IV;

The thyroid tumor is determined as a late-stage thyroid cancer if the expression of both Z1 and Z16 with loss of imprinting are grade IV, and/or the expression of both Z1 and Z16 with copy number variation are grade IV.

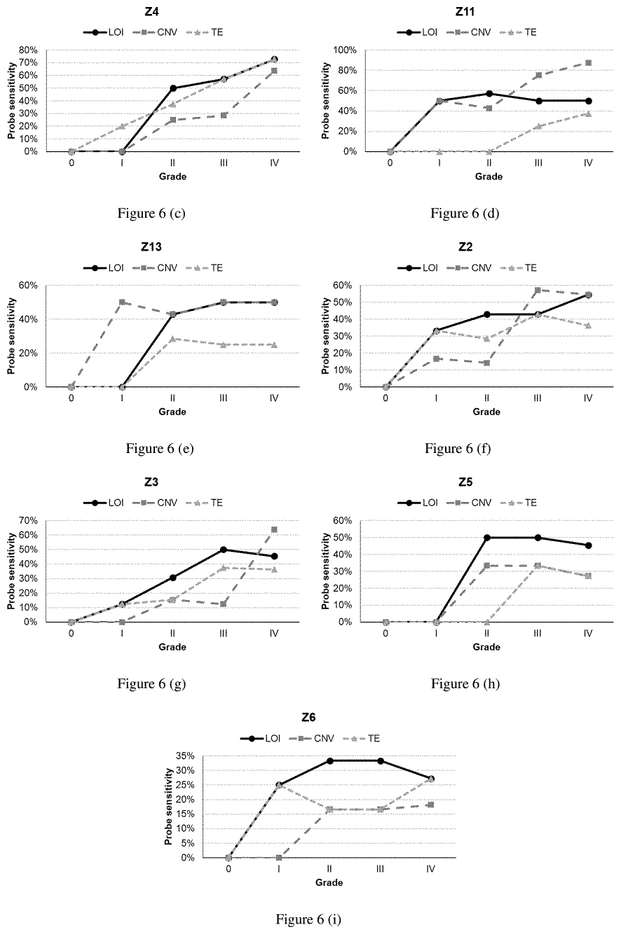

The sensitivity of each imprinted gene for thyroid cancer is shown in FIG. 6(a)-FIG. 6(i):

The loss of imprinting, copy number variation and the total expression of imprinted gene Z1 increased rapidly at cancer potential stage, reached a high sensitivity in early-stage thyroid cancer, and maintained a high level in medium- and late-stage thyroid cancer. The loss of imprinting of imprinted gene Z16 increased rapidly at cancer potential stage, and maintained a high level during the progression of thyroid cancer. The copy number variation and total expression of imprinted gene Z16 increased rapidly in early-stage thyroid cancer, and maintained a high level in medium- and late-stage thyroid cancer.

The loss of imprinting and copy number variation of imprinted gene Z4 started increasing at early-stage thyroid cancer, and increased to a high level gradually at medium-stage and late-stage thyroid cancer. The increase of total expression of imprinted gene Z4 first appeared at cancer potential stage, increased with the progression of thyroid cancer, and reached a high level in late-stage thyroid cancer. The loss of imprinting of imprinted gene Z11 increased rapidly at cancer potential stage, but did not increase any more in the progression from early-stage to late-stage thyroid cancer. The copy number variation of imprinted gene Z11 increased rapidly at cancer potential stage, remained stable in early-stage thyroid cancer, and increased again in medium- and late-stage thyroid cancer. The increase of total expression of imprinted gene Z11 started in medium-stage thyroid cancer, and further increased in late-stage thyroid cancer. The copy number variation of imprinted gene Z13 increased rapidly at cancer potential stage, the loss of imprinting and total expression of Z13 started increasing in early-stage thyroid cancer, but remained stable in medium- and late-stage thyroid cancer.

The loss of imprinting and total expression of imprinted gene Z2 started increasing at cancer potential stage, and increased slowly during the progression of early-stage to late-stage thyroid cancer. The copy number variation of imprinted gene Z2 first appeared at cancer potential stage, stopped increasing in early-stage thyroid cancer, increased again in medium-stage thyroid cancer, and remained stable in late-stage thyroid cancer. The loss of imprinting and increase of total expression of imprinted gene Z3 first appeared at cancer potential stage, increased slowly during the progression of thyroid cancer, while the sensitivities were still not high in late-stage thyroid cancer; The copy number variation of imprinted gene Z3 was quite low before medium-stage thyroid cancer, while increased to a high level rapidly in late-stage thyroid cancer. The loss of imprinting and copy number variation of imprinted gene Z5 increased rapidly in early-stage thyroid cancer, and remained stable in medium- and late-stage thyroid cancer. The increase of total expression of imprinted gene Z5 first appeared in medium-stage thyroid cancer, and remained stable in late-stage thyroid cancer. The loss of imprinting and increase of total expression of imprinted gene Z6 first appeared at cancer potential stage, and maintained a low level during the progression of thyroid cancer. The copy number variation of imprinted gene Z6 first appeared in early-stage thyroid cancer, and maintained a low level in medium- and late-stage thyroid cancer.

Example 3 Imprinted Gene Analysis for 18 breast aspiration cell samples

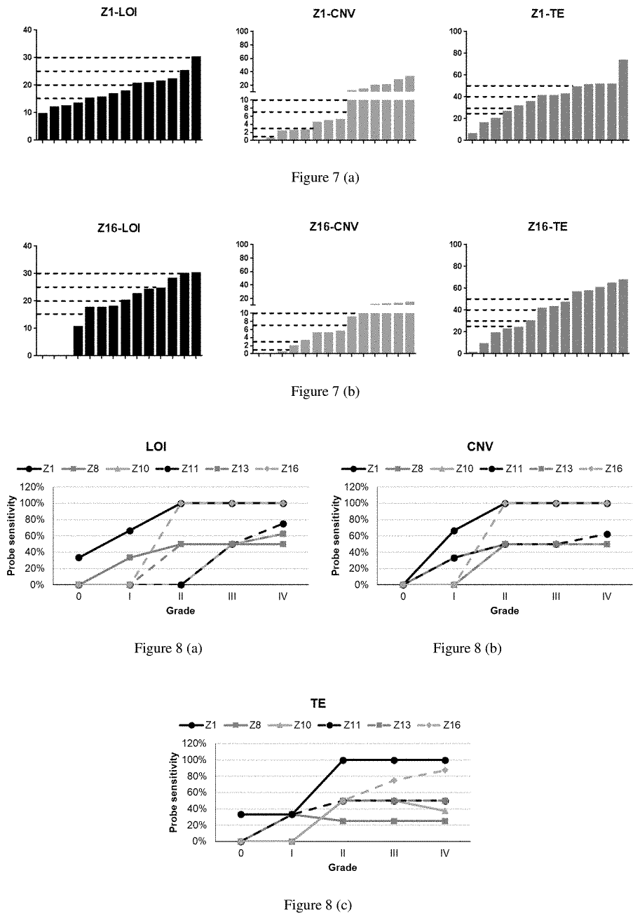

The breast aspiration cell samples were obtained by needle aspiration. Other methods were the same as in Example 1. The results are shown in FIGS. 7(a)-7(b), FIGS. 8(a)-8(f), and FIGS. 9(a)-9 (k).

As shown in FIG. 7(a), for the imprinted gene Z1, grade 0 is defined as when the expression of the imprinted gene with loss of imprinting is less than 15%, and/or the expression of imprinted gene with copy number variation is less than 1%, and/or the total expression of imprinted gene is less than 25%. Grade I is defined as when the expression of the imprinted gene with loss of imprinting is between 15-20%, and/or the expression of imprinted gene with copy number variation is between 1-3%, and/or the total expression of imprinted gene is between 25-30%. Grade II is defined as when the expression of the imprinted gene with loss of imprinting is between 20-25%, and/or the expression of imprinted gene with copy number variation is between 3-7%, and/or the total expression of imprinted gene is between 30-40%. Grade III is defined as when the expression of the imprinted gene with loss of imprinting is between 25-30%, and/or the expression of imprinted gene with copy number variation is between 7-10%, and/or the total expression of imprinted gene is between 40-50%. Grade IV is defined as when the expression of the imprinted gene with loss of imprinting is more than 30%, and/or the expression of imprinted gene with copy number variation is more than 10%, and/or the total expression of imprinted gene is more than 50%.

As shown in FIG. 7(b), for the imprinted gene Z16, grade 0 is defined as when the expression of the imprinted gene with loss of imprinting is less than 15%, and/or the expression of imprinted gene with copy number variation is less than 1%, and/or the total expression of imprinted gene is less than 25%. Grade I is defined as when the expression of the imprinted gene with loss of imprinting is between 15-20%, and/or the expression of imprinted gene with copy number variation is between 1-3%, and/or the total expression of imprinted gene is between 25-30%. Grade II is defined as when the expression of the imprinted gene with loss of imprinting is between 20-25%, and/or the expression of imprinted gene with copy number variation is between 3-7%, and/or the total expression of imprinted gene is between 30-40%. Grade III is defined as when the expression of the imprinted gene with loss of imprinting is between 25-30%, and/or the expression of imprinted gene with copy number variation is between 7-10%, and/or the total expression of imprinted gene is between 40-50%. Grade IV is defined as when the expression of the imprinted gene with loss of imprinting is more than 30%, and/or the expression of imprinted gene with copy number variation is more than 10%, and/or the total expression of imprinted gene is more than 50%.

From the comprehensive analysis of these 18 breast aspiration cell samples, it can be concluded that:

The breast tumor is determined as a benign tumor, if the expression of both Z1 and Z16 with loss of imprinting are lower than grade I; if the expression of only one of Z1 and Z16 with loss of imprinting is grade I; if the expression of only one of Z1 and Z16 with copy number variation is grade I;

The breast tumor is determined as breast cancer potential, if the expression of both Z1 and Z16 with loss of imprinting are grade I; if the expression of both Z1 and Z16 with copy number variation are grade I; if the expression of only one of Z1 and Z16 with loss of imprinting is grade I and the expression of only one of Z1 and Z16 with copy number variation is grade I; if the expression of only one of Z1 and Z16 with loss of imprinting is grade II; if the expression of only one of Z1 and Z16 with copy number variation is grade II;

The breast tumor is determined as an early-stage breast cancer; if the expression of both Z1 and Z16 with loss of imprinting are grade II, and/or the expression of both Z1 and Z16 with copy number variation are grade II; if the expression of only one of Z1 and Z16 with loss of imprinting is grade II and the expression of only one of Z1 and Z16 with copy number variation is grade II; if the expression of only one of Z1 and Z16 with loss of imprinting is grade III; if the expression of only one of Z1 and Z16 with copy number variation is grade III;

The breast tumor is determined as a medium-stage breast cancer; if the expression of both Z1 and Z16 with loss of imprinting are grade III; if the expression of both Z1 and Z16 with copy number variation are grade III; if the expression of only one of Z1 and Z16 with loss of imprinting is grade III and the expression of only one of Z1 and Z16 with copy number variation is grade III; if the expression of only one of Z1 or Z16 with loss of imprinting is grade IV; if the expression of only one of Z1 or Z16 with copy number variation is grade IV;

The breast tumor is determined as a late-stage breast cancer if the expression of both Z1 and Z16 with loss of imprinting are grade IV, and/or the expression of both Z1 and Z16 with copy number variation are grade IV.

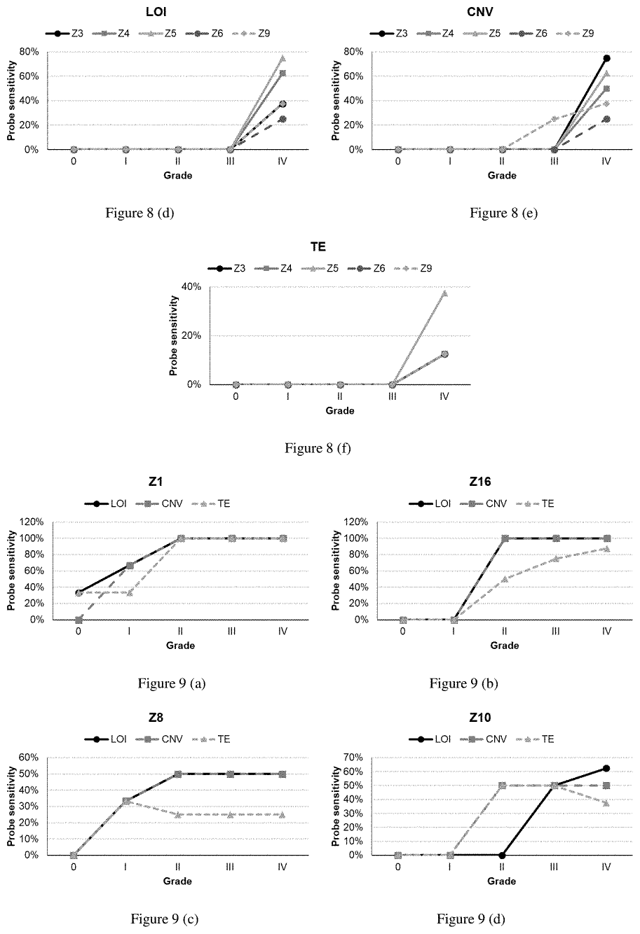

From FIGS. 8(a)-8(f), it can be observed that the sensitivity for breast cancer and the intensity and status of loss of imprinting in breast cancer is different between imprinted genes Z1, Z2, Z3, Z4, Z5, Z6, Z7, Z8, Z9, Z10, Z11, Z12, Z13, Z14, Z15, and Z16. Among them, Z1, Z16, Z8, Z10, Z11, and Z13 show higher sensitivities.

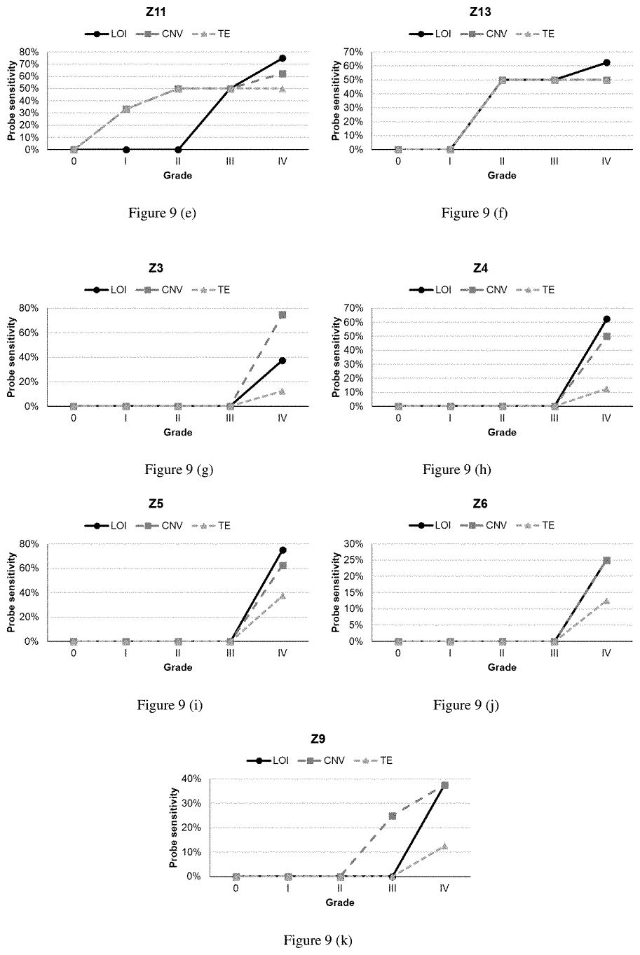

The sensitivity of each imprinted gene for breast cancer is shown in FIGS. 9(a)-9(k):

The loss of imprinting and copy number variation of imprinted gene Z1 increased rapidly at cancer potential stage, achieved a high sensitivity in early-stage breast cancer, and remained high in medium- and late-stage breast cancer; The total expression of imprinted gene Z1 was not high at cancer potential stage, but increased to a high level rapidly in early-stage breast cancer, and maintained a high level in medium- and late-stage breast cancer. The loss of imprinting and copy number variation of imprinted gene Z16 increased rapidly in early-stage breast cancer, and remained high in medium- and late-stage breast cancer; The increase of total expression of imprinted gene Z16 first appeared in early-stage breast cancer, and further increased to a high level in medium- and late-stage breast cancer.

The loss of imprinting and copy number variation of imprinted gene Z8 first appeared at cancer potential stage, further increased in early-stage breast cancer, and remained stable in medium- and late-stage breast cancer; The increase of total expression of imprinted gene Z8 first appeared at cancer potential stage, but showed no further increase in the progression of breast cancer. The copy number variation and total expression of imprinted gene Z10 increased rapidly in early-stage breast cancer, but showed no further increase in medium- and late-stage breast cancer; The loss of imprinting of imprinted gene Z10 first appeared in medium-stage breast cancer, and showed a slight increase in late-stage breast cancer. The copy number variation and increase of total expression of imprinted gene Z11 first appeared at cancer potential stage, while the increasing speed slowed down in early-stage breast cancer, and remained stable in medium- and late-stage breast cancer. The loss of imprinting of imprinted gene Z11 first appeared in medium-stage breast cancer, and increased to a high level in late-stage breast cancer. The loss of imprinting, copy number variation and total expression of imprinted gene Z13 increased rapidly in early-stage breast cancer, and remained stable in medium- and late-stage breast cancer. The loss of imprinting, copy number variation and increase of total expression of imprinted gene Z3 first appeared in late-stage breast cancer, wherein the level of copy number variation was relatively high, while the level of loss of imprinting and total expression were relatively low. The loss of imprinting, copy number variation and increase of total expression of imprinted genes Z4 and Z5 first appeared in late-stage breast cancer, wherein the level of loss of imprinting and copy number variation were relatively high, while the level of total expression was relatively low. The loss of imprinting, copy number variation and increase of total expression of imprinted gene Z6 first appeared in late-stage breast cancer, but their levels were not high. The copy number variation of imprinted gene Z9 first appeared in medium-stage breast cancer, and further increased in late-stage breast cancer. The loss of imprinting and increase of total expression of imprinted gene Z9 first appeared in late-stage breast cancer, but their levels were not high.

### EXAMPLE 4

### Imprinted Gene Analysis for 21 Pancreas Aspiration Cell Samples

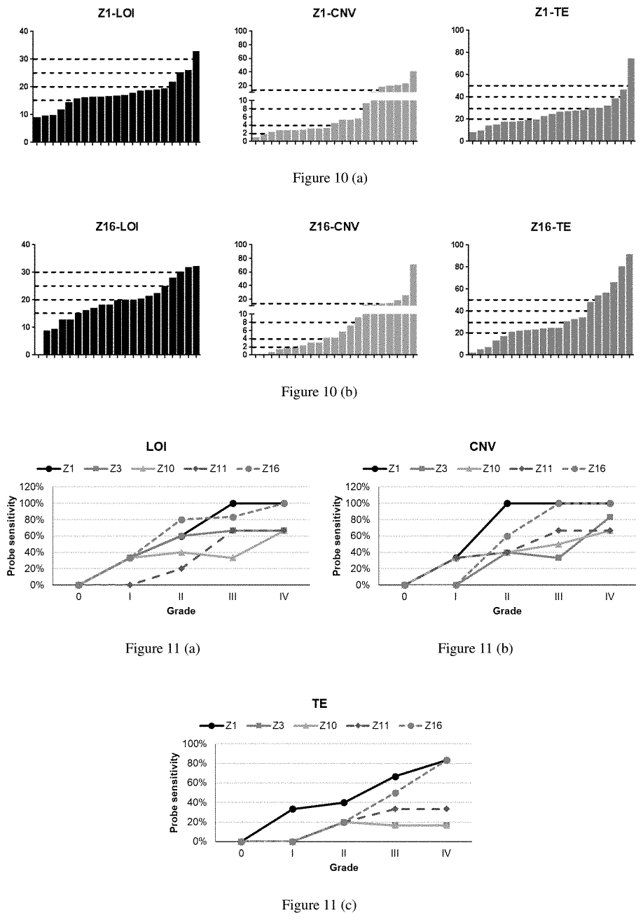

21 pancreatic aspiration cell samples were obtained by needle aspiration. Other methods were the same as in Example 1. The results are shown in FIGS. 10(a)-10(b), FIGS. 11(a)-11(i) and FIGS. 12(a)-12(o).

As shown in FIG. 10(a), for the imprinted gene Z1, grade 0 is defined as when the expression of the imprinted gene with loss of imprinting is less than 15%, and/or the expression of imprinted gene with copy number variation is less than 2%, and/or the total expression of imprinted gene is less than 20%. Grade I is defined as when the expression of the imprinted gene with loss of imprinting is between 15-20%, and/or the expression of imprinted gene with copy number variation is between 2-4%, and/or the total expression of imprinted gene is between 20-30%. Grade II is defined as when the expression of the imprinted gene with loss of imprinting is between 20-25%, and/or the expression of imprinted gene with copy number variation is between 4-8%, and/or the total expression of imprinted gene is between 30-40%. Grade III is defined as when the expression of the imprinted gene with loss of imprinting is between 25-30%, and/or the expression of imprinted gene with copy number variation is between 8-12%, and/or the total expression of imprinted gene is between 40-50%. Grade IV is defined as when the expression of the imprinted gene with loss of imprinting is more than 30%, and/or the expression of imprinted gene with copy number variation is more than 12%, and/or the total expression of imprinted gene is more than 50%.

As shown in FIG. 10(b), for the imprinted gene Z16, grade 0 is defined as when the expression of the imprinted gene with loss of imprinting is less than 15%, and/or the expression of imprinted gene with copy number variation is less than 2%, and/or the total expression of imprinted gene is less than 20%. Grade I is defined as when the expression of the imprinted gene with loss of imprinting is between 15-20%, and/or the expression of imprinted gene with copy number variation is between 2-4%, and/or the total expression of imprinted gene is between 20-30%. Grade II is defined as when the expression of the imprinted gene with loss of imprinting is between 20-25%, and/or the expression of imprinted gene with copy number variation is between 4-8%, and/or the total expression of imprinted gene is between 30-40%. Grade III is defined as when the expression of the imprinted gene with loss of imprinting is between 25-30%, and/or the expression of imprinted gene with copy number variation is between 8-12%, and/or the total expression of imprinted gene is between 40-50%. Grade IV is defined as when the expression of the imprinted gene with loss of imprinting is more than 30%, and/or the expression of imprinted gene with copy number variation is more than 12%, and/or the total expression of imprinted gene is more than 50%.

From the comprehensive analysis of these 21 pancreatic aspiration cell samples, it can be concluded that:

The pancreatic tumor is determined as a benign tumor, if the expression of both Z1 and Z16 with loss of imprinting are lower than grade I; if the expression of only one of Z1 and Z16 with loss of imprinting is grade I;

if the expression of only one of Z1 and Z16 with copy number variation is grade I;

The pancreatic tumor is determined as pancreatic cancer potential, if the expression of both Z1 and Z16 with loss of imprinting are grade I; if the expression of both Z1 and Z16 with copy number variation are grade I; if the expression of only one of Z1 and Z16 with loss of imprinting is grade I and the expression of only one of Z1 and Z16 with copy number variation is grade I; if the expression of only one of Z1 and Z16 with loss of imprinting is grade II; if the expression of only one of Z1 and Z16 with copy number variation is grade II;

The pancreatic tumor is determined as an early-stage pancreatic cancer; if the expression of both Z1 and Z16 with loss of imprinting are grade II, and/or the expression of both Z1 and Z16 with copy number variation are grade II; if the expression of only one of Z1 and Z16 with loss of imprinting is grade II and the expression of only one of Z1 and Z16 with copy number variation is grade II; if the expression of only one of Z1 and Z16 with loss of imprinting is grade III; if the expression of only one of Z1 and Z16 with copy number variation is grade III;

The pancreatic tumor is determined as a medium-stage pancreatic cancer; if the expression of both Z1 and Z16 with loss of imprinting are grade III; if the expression of both Z1 and Z16 with copy number variation are grade III; if the expression of only one of Z1 and Z16 with loss of imprinting is grade III and the expression of only one of Z1 and Z16 with copy number variation is grade III; if the expression of only one of Z1 or Z16 with loss of imprinting is grade IV; if the expression of only one of Z1 or Z16 with copy number variation is grade IV;

The pancreatic tumor is determined as a late-stage pancreatic cancer if the expression of both Z1 and Z16 with loss of imprinting are grade IV, and/or the expression of both Z1 and Z16 with copy number variation are grade IV.

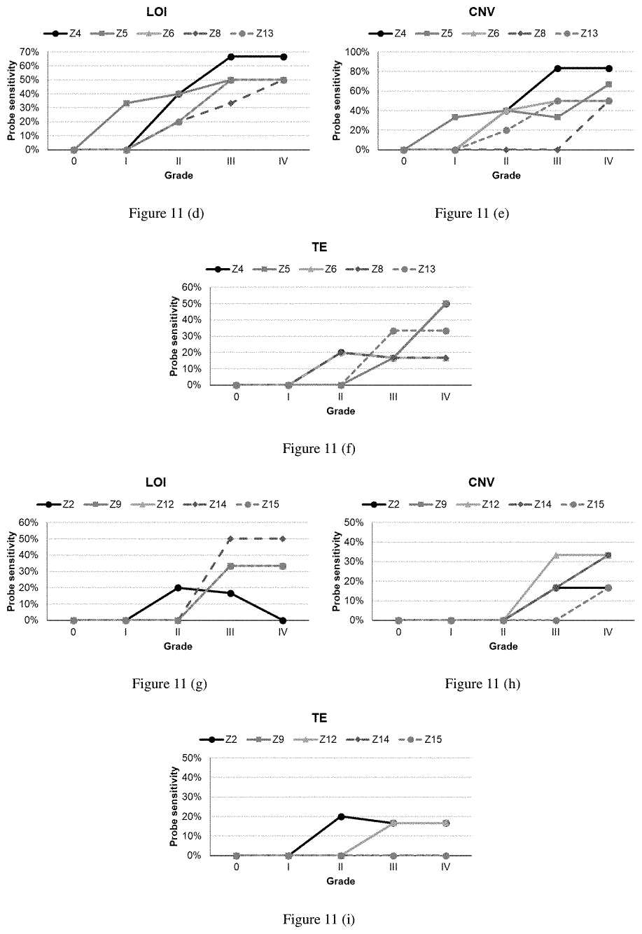

From FIGS. 11(a)-11(i), it can be observed that the sensitivity for pancreatic cancer and the intensity and status of loss of imprinting in pancreatic cancer is different between imprinted genes Z1, Z2, Z3, Z4, Z5, Z6, Z7, Z8, Z9, Z10, Z11, Z12, Z13, Z14, Z15, and Z16. Among them, Z1, Z16, Z3, Z10, Z11 show higher sensitivities.

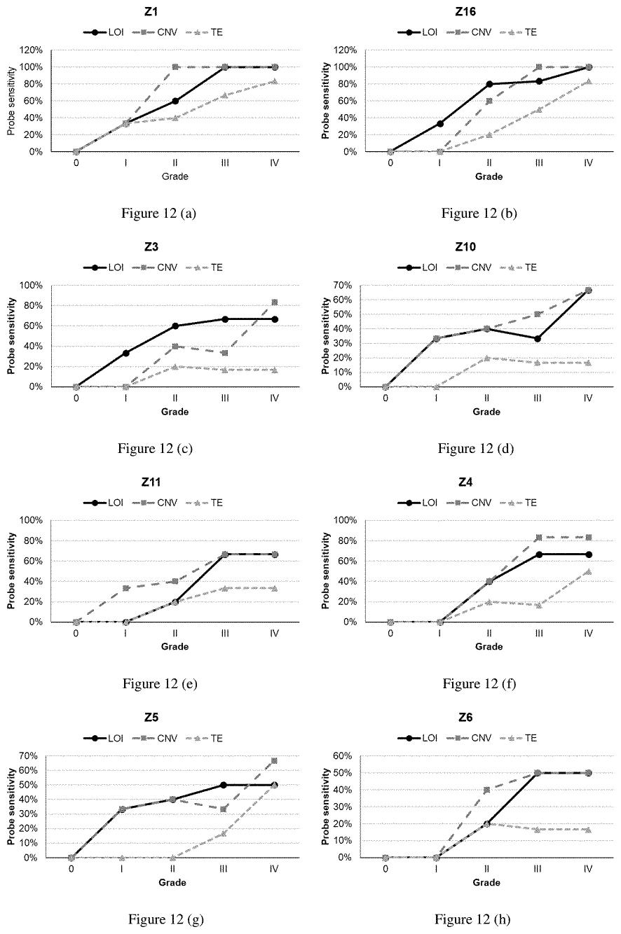

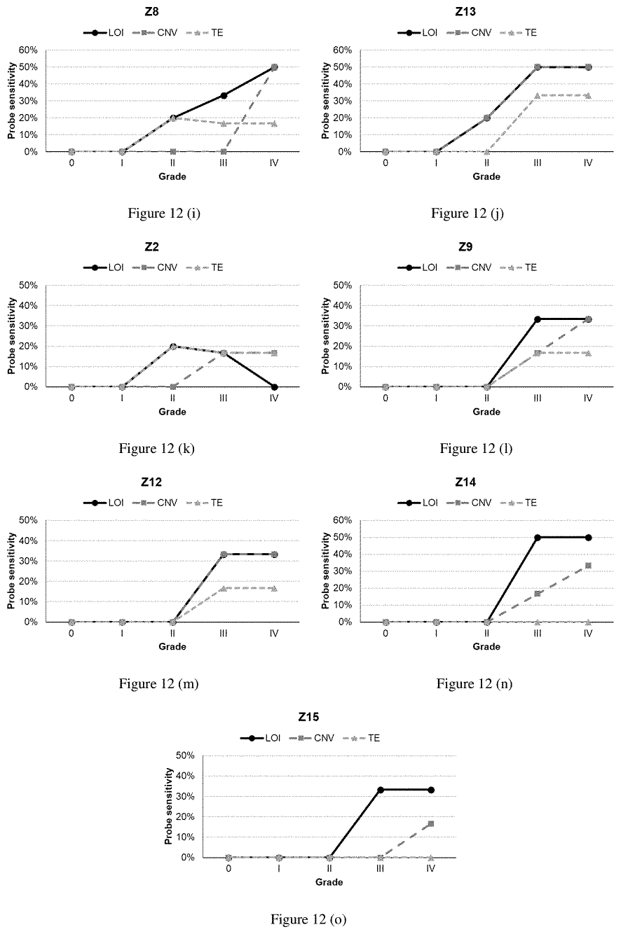

The sensitivity of each imprinted gene for pancreatic cancer is shown in FIGS. 12(a)-12(o):

The loss of imprinting, copy number variation and increase of total expression of imprinted gene Z1 first appeared at cancer potential stage, among which the copy number variation increased in a fastest speed, reached a high level in early-stage pancreatic cancer and remained stable afterwards. The loss of imprinting also increased in a relatively fast speed, and reached a high level in medium-stage pancreatic cancer. The total expression increased slowly, and finally reached a high level in late-stage pancreatic cancer. The loss of imprinting of imprinted gene Z16 first appeared at cancer potential stage, increased gradually during the progression of pancreatic cancer, and reached a high level in late-stage pancreatic cancer. The copy number variation of imprinted gene Z16 first appeared in early-stage pancreatic cancer, increased rapidly in medium-stage pancreatic cancer, and maintained a high level in late-stage pancreatic cancer; The increase of total expression of imprinted gene Z16 first appeared in early-stage pancreatic cancer, kept increasing during the progression of pancreatic cancer, and reached a high level in late-stage pancreatic cancer.

The loss of imprinting of imprinted gene Z3 first appeared at cancer potential stage, kept increasing during the progression of pancreatic cancer, while the speed of increase slowed down in medium- and late-stage pancreatic cancer. The copy number variation of imprinted gene Z3 first appeared in early-stage pancreatic cancer, showed no significant increase in medium-stage pancreatic cancer, but increased again to a high level in late-stage pancreatic cancer. The increase of total expression of imprinted gene Z3 first appeared in early-stage pancreatic cancer, but maintained a low level in medium- and late-stage pancreatic cancer. The loss of imprinting and copy number variation of imprinted gene Z10 first appeared at cancer potential stage, showed no significant increase in early-stage and medium-stage pancreatic cancer, but increased again to a high level in late-stage pancreatic cancer; The increase of total expression of imprinted gene Z10 first appeared in early-stage pancreatic cancer, but maintained a low level in medium- and late-stage pancreatic cancer. The copy number variation of imprinted gene Z11 first appeared at cancer potential stage, increased gradually in early-stage and medium-stage pancreatic cancer, and maintained a high level in late-stage pancreatic cancer. The loss of imprinting and increase of total expression of imprinted gene Z11 first appeared in early-stage pancreatic cancer, among which the loss of imprinting increased at a relatively high speed, and maintained a high level in medium- and late-stage pancreatic cancer, while the total expression increased slowly, and its level was still not high in medium- and late-stage pancreatic cancer. The loss of imprinting and copy number variation of imprinted gene Z4 first appeared in early-stage pancreatic cancer, increased rapidly in medium-stage pancreatic cancer, and maintained a high level in late-stage pancreatic cancer. The increase of total expression of imprinted gene Z4 first appeared in early-stage pancreatic cancer, showed no significant increase in medium-stage pancreatic cancer, while increased again in late-stage pancreatic cancer. The loss of imprinting and copy number variation of imprinted gene Z5 first appeared at cancer potential stage, loss of imprinting showed no significant increase in the progression from early-stage to late-stage pancreatic cancer; copy number variation showed no significant increase in the progression from early-stage to medium-stage pancreatic cancer, but increased again to a high level in late-stage pancreatic cancer; The increase of total expression of imprinted gene Z5 first appeared in medium-stage pancreatic cancer, and continued increasing in late-stage pancreatic cancer but its level was still low. The loss of imprinting and copy number variation of imprinted gene Z6 first appeared in early-stage pancreatic cancer, increased during the progression of pancreatic cancer, and reached a high level in late-stage pancreatic cancer; The increase of total expression of imprinted gene Z6 first appeared in early-stage pancreatic cancer, but maintained a low level in medium- and late-stage pancreatic cancer. The loss of imprinting of imprinted gene Z8 first appeared in early-stage pancreatic cancer, increased slowly during the progression of pancreatic cancer, and reached a high level in late-stage pancreatic cancer. The copy number variation of imprinted gene Z8 appeared in late-stage pancreatic cancer and with a high level. The increase of total expression of imprinted gene Z8 first appeared in early-stage pancreatic cancer, but maintained a low level in medium- and late-stage pancreatic cancer. The loss of imprinting and copy number variation of imprinted gene Z13 first appeared in early-stage pancreatic cancer, increased rapidly in medium-stage pancreatic cancer, and remained stable in late-stage pancreatic cancer. The increase of total expression of imprinted gene Z13 first appeared in medium-stage pancreatic cancer, but stopped increasing in late-stage pancreatic cancer. The loss of imprinting and increase of total expression of imprinted gene Z2 first appeared in early-stage pancreatic cancer. The copy number variation of imprinted gene Z2 first appeared in medium-stage pancreatic cancer, but maintained a low level in medium- and late-stage pancreatic cancer. The loss of imprinting of imprinted gene Z9 first appeared in medium-stage pancreatic cancer, increased slowly during the progression of pancreatic cancer, and its level was still not high in late-stage pancreatic cancer; The copy number variation of imprinted gene Z9 first appeared in medium-stage pancreatic cancer, and kept increasing in late-stage pancreatic cancer, but its level was not high. The increase of total expression of imprinted gene Z9 first appeared in medium-stage pancreatic cancer, but stopped increasing in late-stage pancreatic cancer. The loss of imprinting, copy number variation and increase of total expression of imprinted gene Z12 first appeared in medium-stage pancreatic cancer, but stopped increasing in late-stage pancreatic cancer. The loss of imprinting of imprinted gene Z14 increased rapidly in medium-stage pancreatic cancer, and maintained a high level in late-stage pancreatic cancer. The copy number variation of imprinted gene Z14 first appeared in medium-stage pancreatic cancer, and continued increasing in late-stage pancreatic cancer, but its level was not high; Imprinted gene Z14 did not show significant increase of the total expression during the progression of pancreatic cancer. The loss of imprinting of imprinted gene Z15 first appeared in medium-stage pancreatic cancer, but stopped increasing in late-stage pancreatic cancer. The copy number variation of imprinted gene Z15 first appeared in late-stage pancreatic cancer, but its level was not high; Imprinted gene Z15 did not show significant increase of total expression in the progression of pancreatic cancer.

### EXAMPLE 5

### Imprinted Gene Analysis for 23 Bronchial Brush Cell Samples

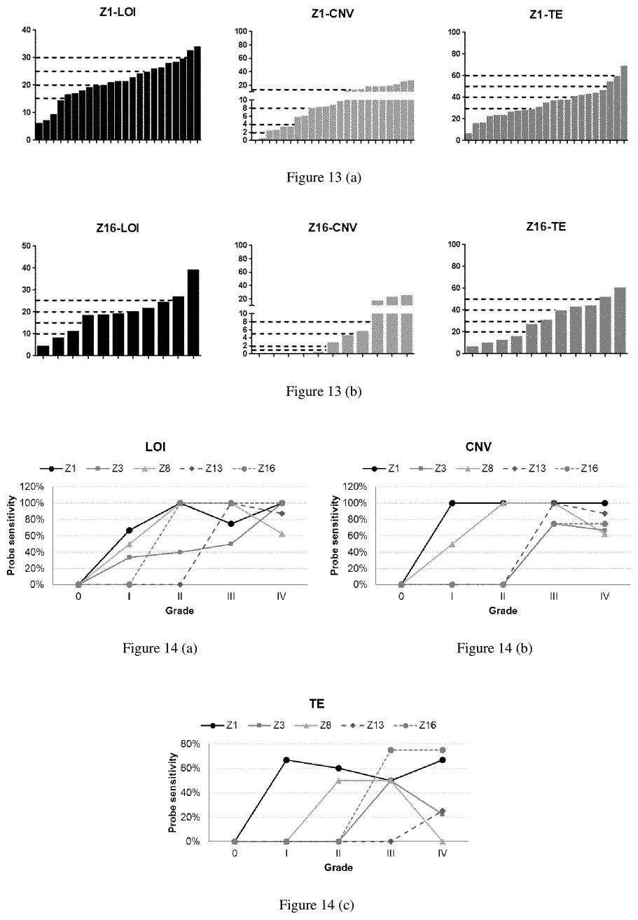

The lung tumor cell samples were obtained through bronchial brushing. Other methods are the same as in Example 1. The results are shown in FIGS. 13(a)-13(b), FIGS. 14(a)-14(f) and FIGS. 15(a)-15(h).

As shown in FIG. 13(a), for the imprinted gene Z1, grade 0 is defined as when the expression of the imprinted gene with loss of imprinting is less than 15%, and/or the expression of imprinted gene with copy number variation is less than 2%, and/or the total expression of imprinted gene is less than 30%. Grade I is defined as when the expression of the imprinted gene with loss of imprinting is between 15-20%, and/or the expression of imprinted gene with copy number variation is between 2-4%, and/or the total expression of imprinted gene is between 30-40%. Grade II is defined as when the expression of the imprinted gene with loss of imprinting is between 20-25%, and/or the expression of imprinted gene with copy number variation is between 4-8%, and/or the total expression of imprinted gene is between 40-50%. Grade III is defined as when the expression of the imprinted gene with loss of imprinting is between 25-30%, and/or the expression of imprinted gene with copy number variation is between 8-12%, and/or the total expression of imprinted gene is between 50-60%. Grade IV is defined as when the expression of the imprinted gene with loss of imprinting is more than 30%, and/or the expression of imprinted gene with copy number variation is more than 12%, and/or the total expression of imprinted gene is more than 60%.

As shown in FIG. 13(b), for the imprinted gene Z16, grade 0 is defined as when the expression of the imprinted gene with loss of imprinting is less than 10%, and/or the expression of imprinted gene with copy number variation is less than 1%, and/or the total expression of imprinted gene is less than 25%. Grade I is defined as when the expression of the imprinted gene with loss of imprinting is between 10-15%, and/or the expression of imprinted gene with copy number variation is between 1-2%, and/or the total expression of imprinted gene is between 25-30%. Grade II is defined as when the expression of the imprinted gene with loss of imprinting is between 15-20%, and/or the expression of imprinted gene with copy number variation is between 2-5%, and/or the total expression of imprinted gene is between 30-40%. Grade III is defined as when the expression of the imprinted gene with loss of imprinting is between 20-25%, and/or the expression of imprinted gene with copy number variation is between 5-8%, and/or the total expression of imprinted gene is between 40-50%. Grade IV is defined as when the expression of the imprinted gene with loss of imprinting is more than 25%, and/or the expression of imprinted gene with copy number variation is more than 8%, and/or the total expression of imprinted gene is more than 50%.

From the comprehensive analysis of these 23 bronchial brushing cell samples, it can be concluded that:

The lung tumor is determined as a benign tumor, if the expression of both Z1 and Z16 with loss of imprinting are lower than grade I; if the expression of only one of Z1 and Z16 with loss of imprinting is grade I; if the expression of only one of Z1 and Z16 with copy number variation is grade I;

The lung tumor is determined as lung cancer potential, if the expression of both Z1 and Z16 with loss of imprinting are grade I; if the expression of both Z1 and Z16 with copy number variation are grade I; if the expression of only one of Z1 and Z16 with loss of imprinting is grade I and the expression of only one of Z1 and Z16 with copy number variation is grade I; if the expression of only one of Z1 and Z16 with loss of imprinting is grade II; if the expression of only one of Z1 and Z16 with copy number variation is grade II;

The lung tumor is determined as an early-stage lung cancer; if the expression of both Z1 and Z16 with loss of imprinting are grade II, and/or the expression of both Z1 and Z16 with copy number variation are grade II; if the expression of only one of Z1 and Z16 with loss of imprinting is grade II and the expression of only one of Z1 and Z16 with copy number variation is grade II; if the expression of only one of Z1 and Z16 with loss of imprinting is grade III; if the expression of only one of Z1 and Z16 with copy number variation is grade III;

The lung tumor is determined as a medium-stage lung cancer; if the expression of both Z1 and Z16 with loss of imprinting are grade III; if the expression of both Z1 and Z16 with copy number variation are grade III; if the expression of only one of Z1 and Z16 with loss of imprinting is grade III and the expression of only one of Z1 and Z16 with copy number variation is grade III; if the expression of only one of Z1 or Z16 with loss of imprinting is grade IV; if the expression of only one of Z1 or Z16 with copy number variation is grade IV;

The lung tumor is determined as a late-stage lung cancer if the expression of both Z1 and Z16 with loss of imprinting are grade IV, and/or the expression of both Z1 and Z16 with copy number variation are grade IV.

From FIG. 14 (a)-FIG. 14 (f), it can be observed that the sensitivity for lung cancer and the intensity and status of loss of imprinting in lung cancer is different between imprinted genes Z1, Z2, Z3, Z4, Z5, Z6, Z7, Z8, Z9, Z10, Z11, Z12, Z13, Z14, Z15, and Z16. Among them, Z1, Z16, Z3, Z8, Z13 show higher sensitivities.

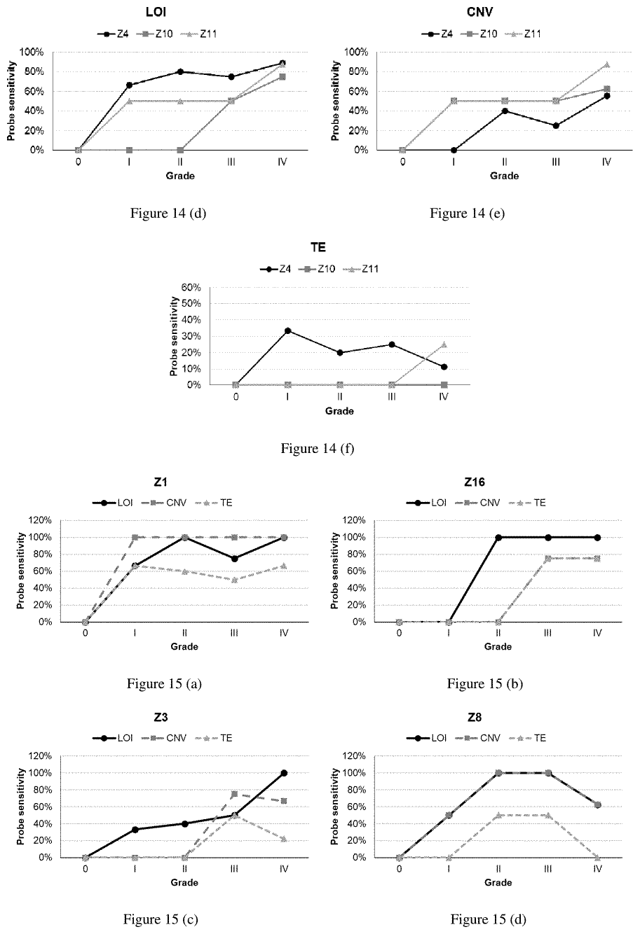

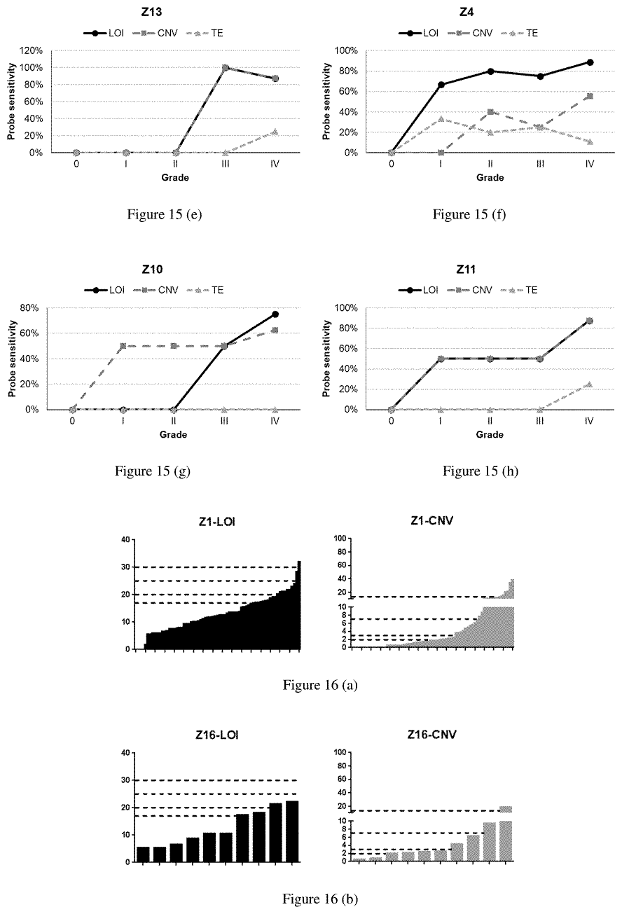

The sensitivity of each imprinted gene for lung cancer is shown in FIG. 15 (a)-FIG. 15 (h):

The loss of imprinting and copy number variation of imprinted gene Z1 increased rapidly at cancer potential stage, and maintained a high level during the progression of lung cancer; The total expression of imprinted gene Z1 increased rapidly at cancer potential stage, while stopped increasing in the progression from early-stage to late-stage lung cancer. The loss of imprinting of imprinted gene Z16 increased rapidly in early-stage lung cancer, and maintained a high level in medium- and late-stage lung cancer; The copy number variation and total expression of imprinted gene Z16 increased rapidly in medium-stage lung cancer, and maintained a high level in late-stage lung cancer.

The loss of imprinting of imprinted gene Z3 first appeared at cancer potential stage, showed no significant increase in early-stage and medium-stage lung cancer, while increased to a high level rapidly in late-stage lung cancer; The copy number variation of imprinted gene Z3 increased to a high level rapidly in medium-stage lung cancer, and remained stable in late-stage lung cancer; The increase of total expression of imprinted gene Z3 first appeared in medium-stage lung cancer, but the increase was not significant in late-stage lung cancer. The loss of imprinting and copy number variation of imprinted gene Z8 first appeared at cancer potential stage, further increase in early-stage lung cancer, and maintained a high level in medium-stage lung cancer, but decreased in late-stage lung cancer. The increase of total expression of imprinted gene Z8 appeared in early-stage and medium-stage lung cancer, but returned to a low level in late-stage lung cancer. The loss of imprinting and copy number variation of imprinted gene Z13 increased rapidly in medium-stage lung cancer, and maintained a high level in late-stage lung cancer. The total expression of imprinted gene Z13 slightly increased in late-stage lung cancer. The loss of imprinting of imprinted gene Z4 increased rapidly at cancer potential stage, and maintained a high level from early-stage to late-stage lung cancer. The copy number variation of imprinted gene Z4 first appeared in early-stage lung cancer, showed no significant increase in medium-stage lung cancer, while increased again to a high level in late-stage lung cancer. The increase of total expression of imprinted gene Z4 first appeared at cancer potential stage, but stopped increasing in the progression of lung cancer. The copy number variation of imprinted gene Z10 increased rapidly at cancer potential stage, and maintained a high level from early-stage to late-stage lung cancer; The loss of imprinting of imprinted gene Z10 increased rapidly in medium-stage lung cancer, and reached a high level in late-stage lung cancer. During the progression of lung cancer, imprinted gene Z10 showed no significant increase of total expression. The loss of imprinting and copy number variation of imprinted gene Z11 increased rapidly at cancer potential stage, and showed no significant increase in early-stage and medium-stage lung cancer, but increased again to a high level in late-stage lung cancer; The total expression of imprinted gene Z11 showed a slight increase only in late-stage lung cancer.

### EXAMPLE 6

### Imprinted Gene Analysis for 70 Urine Exfoliated Cell Samples

The cell samples were obtained from the urine of urinary system tumor patients. Other methods were the same as in Example 1. The results are shown in FIGS. 16(a)-16(b), FIGS. 17(a)-17(d) and FIGS. 18(a)-18(k).

As shown in FIG. 16 (a), for the imprinted gene Z1, grade 0 is defined as when the expression of the imprinted gene with loss of imprinting is less than 17%, and/or the expression of imprinted gene with copy number variation is less than 2%. Grade I is defined as when the expression of the imprinted gene with loss of imprinting is between 17-20%, and/or the expression of imprinted gene with copy number variation is between 2-3%. Grade II is defined as when the expression of the imprinted gene with loss of imprinting is between 20-25%, and/or the expression of imprinted gene with copy number variation is between 3-7%. Grade III is defined as when the expression of the imprinted gene with loss of imprinting is between 25-30%, and/or the expression of imprinted gene with copy number variation is between 7-12%. Grade IV is defined as when the expression of the imprinted gene with loss of imprinting is more than 30%, and/or the expression of imprinted gene with copy number variation is more than 12%.

As shown in FIG. 16 (b), for the imprinted gene Z16, grade 0 is defined as when the expression of the imprinted gene with loss of imprinting is less than 17%, and/or the expression of imprinted gene with copy number variation is less than 2%. Grade I is defined as when the expression of the imprinted gene with loss of imprinting is between 17-20%, and/or the expression of imprinted gene with copy number variation is between 2-3%. Grade II is defined as when the expression of the imprinted gene with loss of imprinting is between 20-25%, and/or the expression of imprinted gene with copy number variation is between 3-7%. Grade III is defined as when the expression of the imprinted gene with loss of imprinting is between 25-30%, and/or the expression of imprinted gene with copy number variation is between 7-12%. Grade IV is defined as when the expression of the imprinted gene with loss of imprinting is more than 30%, and/or the expression of imprinted gene with copy number variation is more than 12%.

From the comprehensive analysis of these 70 urine exfoliated cell samples, it can be concluded that:

The urinary system tumor is determined as a benign tumor, if the expression of both Z1 and Z16 with loss of imprinting are lower than grade I; if the expression of only one of Z1 and Z16 with loss of imprinting is grade I; if the expression of only one of Z1 and Z16 with copy number variation is grade I;

The urinary system tumor is determined as urinary system cancer potential, if the expression of both Z1 and Z16 with loss of imprinting are grade I; if the expression of both Z1 and Z16 with copy number variation are grade I; if the expression of only one of Z1 and Z16 with loss of imprinting is grade I and the expression of only one of Z1 and Z16 with copy number variation is grade I; if the expression of only one of Z1 and Z16 with loss of imprinting is grade II; if the expression of only one of Z1 and Z16 with copy number variation is grade II;

The urinary system tumor is determined as an early-stage urinary system cancer; if the expression of both Z1 and Z16 with loss of imprinting are grade II, and/or the expression of both Z1 and Z16 with copy number variation are grade II; if the expression of only one of Z1 and Z16 with loss of imprinting is grade II and the expression of only one of Z1 and Z16 with copy number variation is grade II; if the expression of only one of Z1 and Z16 with loss of imprinting is grade III; if the expression of only one of Z1 and Z16 with copy number variation is grade III;

The urinary system tumor is determined as a medium-stage urinary system cancer; if the expression of both Z1 and Z16 with loss of imprinting are grade III; if the expression of both Z1 and Z16 with copy number variation are grade III; if the expression of only one of Z1 and Z16 with loss of imprinting is grade III and the expression of only one of Z1 and Z16 with copy number variation is grade III; if the expression of only one of Z1 or Z16 with loss of imprinting is grade IV; if the expression of only one of Z1 or Z16 with copy number variation is grade IV;

The urinary system tumor is determined as a late-stage urinary system cancer if the expression of both Z1 and Z16 with loss of imprinting are grade IV, and/or the expression of both Z1 and Z16 with copy number variation are grade IV.

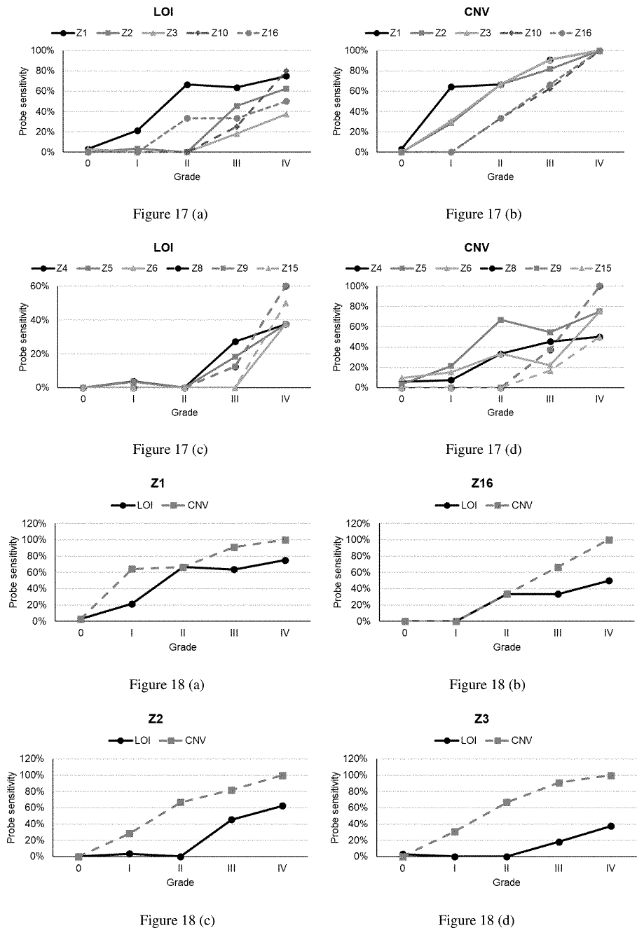

From FIGS. 17(a)-17(d), it can be observed that the sensitivity for urinary system cancer and the intensity and status of loss of imprinting in urinary system cancer is different between imprinted genes Z1, Z2, Z3, Z4, Z5, Z6, Z7, Z8, Z9, Z10, Z11, Z12, Z13, Z14, Z15, and Z16. Among them, Z1, Z16, Z2, Z3, and Z10 show higher sensitivities.

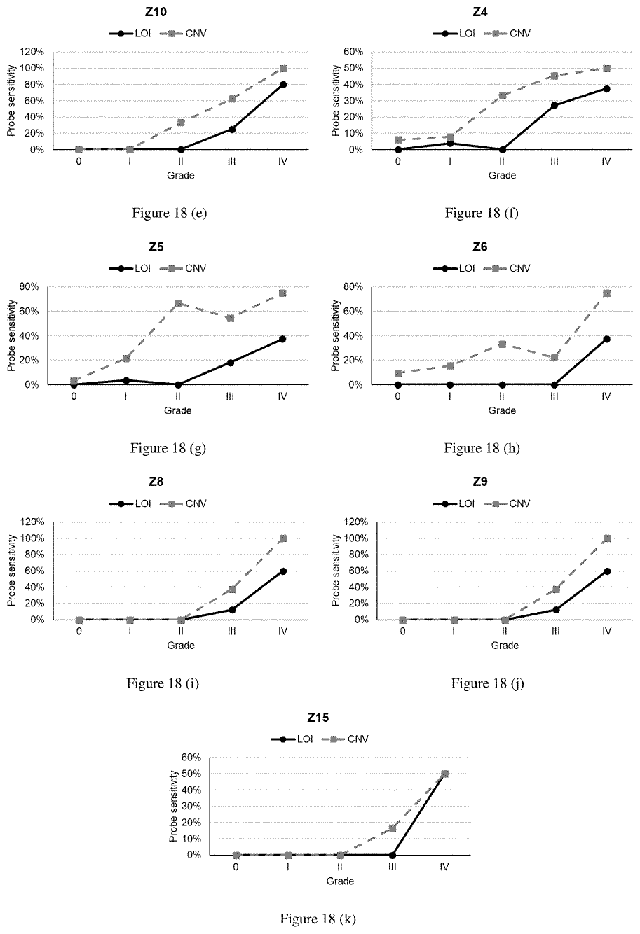

The sensitivity of each imprinted gene for urinary system cancer is shown in FIG. 18(a)-FIG. 18(k):

The loss of imprinting of imprinted gene Z1 first appeared at cancer potential stage, further increased in early-stage urinary system cancer, and maintained a high level in medium- and late-stage urinary system cancer; The copy number variation of imprinted gene Z1 increased rapidly at cancer potential stage, and further increased to a high level in the progression from medium-stage to late-stage urinary system cancer. The loss of imprinting of imprinted gene Z16 first appeared in early-stage urinary system cancer, but was not increasing in medium- and late-stage urinary system cancer. The copy number variation of imprinted gene Z16 first appears in early-stage urinary system cancer, and gradually increased to a high level during the progression of urinary system cancer.

The loss of imprinting of imprinted gene Z2 first appeared in medium-stage urinary system cancer, and reached a high level in late-stage urinary system cancer. The copy number variation of imprinted gene Z2 first appeared at cancer potential stage, and gradually increased to a high level during the progression of urinary system cancer. The loss of imprinting of imprinted gene Z3 first appeared in medium-stage urinary system cancer, and kept increasing in late-stage urinary system cancer, but its level was not high. The copy number variation of imprinted gene Z3 first appeared at cancer potential stage, and gradually increased to a high level during the progression of urinary system cancer. The loss of imprinting of imprinted gene Z10 first appeared in medium-stage urinary system cancer, and increased to a high level rapidly in late-stage urinary system cancer; The copy number variation of imprinted gene Z10 first appeared in early-stage urinary system cancer, and gradually increased to a high level during the progression of urinary system cancer. The loss of imprinting of imprinted gene Z4 first appeared in medium-stage urinary system cancer, and kept increasing in late-stage urinary system cancer, but its level was not high. The copy number variation of imprinted gene Z4 first appeared at cancer potential stage, and gradually increased to a high level during the progression of cancer. The loss of imprinting of imprinted gene Z5 first appeared in medium-stage urinary system cancer, and kept increasing in late-stage cancer, but its level was not high. The copy number variation of imprinted gene Z5 first appeared at cancer potential stage, increased rapidly in early-stage cancer, and maintained a high level in medium- and late-stage urinary system cancer. The copy number variation of imprinted gene Z6 first appeared at cancer potential stage, but did not increase to a high level rapidly until late-stage cancer. The loss of imprinting of imprinted gene Z6 slightly increased in late-stage urinary system cancer. The loss of imprinting and copy number variation of imprinted gene Z8 and Z9 first appeared in medium-stage urinary system cancer, and increased to a high level rapidly in late-stage urinary system cancer. The copy number variation of imprinted gene Z15 first appeared in medium-stage urinary system cancer, and increased to a high level rapidly in late-stage cancer. The loss of imprinting of imprinted gene Z15 showed a large increase in late-stage urinary system cancer.

### EXAMPLE 7

### Imprinted Gene Analysis for Colonoscopy Biopsy Cells

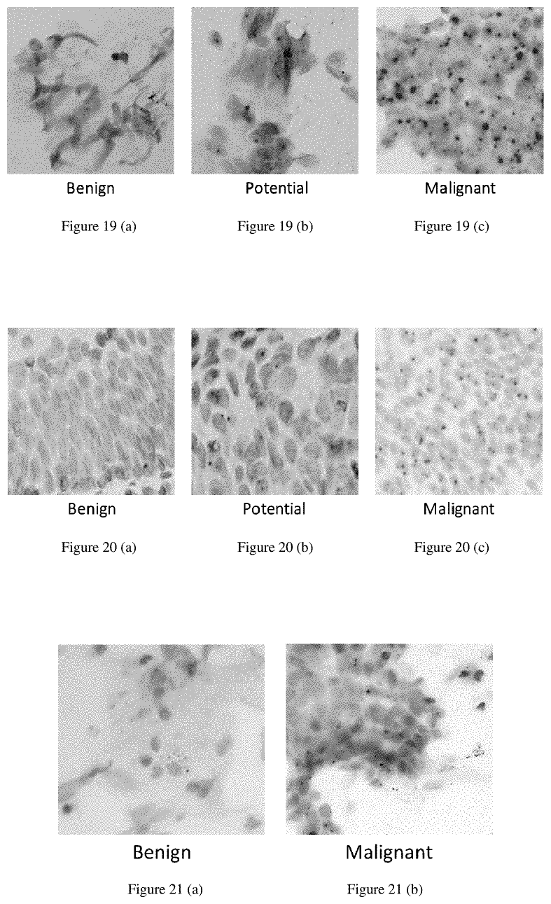

The biopsy samples were obtained under colonoscopy. Other methods were the same as in Example 1. The results are shown in FIG. 19(a)-FIG. 19(c).

As shown in FIG. 19(a)-FIG. 19(c), FIG. 19(a) shows a benign colorectal polyp, FIG. 19(b) shows a malignant potential colorectal tumor, and FIG. 19(c) shows a colorectal cancer. The proportion of cells carrying imprinted genes with loss of imprinting and copy number variation increased gradually with the malignancy of cancer.

### EXAMPLE 8

### Imprinted Gene Analysis for Cystoscopy Biopsy Cells

The biopsy samples were obtained under cystoscopy. Other methods were the same as in Example 1. The results are shown in FIG. 20(a)-FIG. 20(c).

As shown in FIG. 20(a)-FIG. 20(c), FIG. 20(a) shows a benign bladder tumor, FIG. 20(b) shows a malignant potential bladder tumor, and FIG. 20(c) shows a bladder cancer. The proportion of cells carrying imprinted genes with loss of imprinting and copy number variation increased gradually with the malignancy of cancer.

### EXAMPLE 9

### Imprinted Gene Analysis for Sputum Exfoliated Cells

The cell samples were obtained from the sputum of lung tumor patients. Other methods were the same as in Example 1. The results are shown in FIG. 21(a)-FIG. 21(b).

As shown in FIG. 21(a)-FIG. 21(b), FIG. 21(a) shows a benign lung tumor, and FIG. 21(b) shows a lung cancer. The proportion of cells carrying imprinted genes with loss of imprinting and copy number variation increased gradually with the malignancy of cancer.

### EXAMPLE 10

### Imprinted Gene Analysis for Liver Aspiration Biopsy Cells

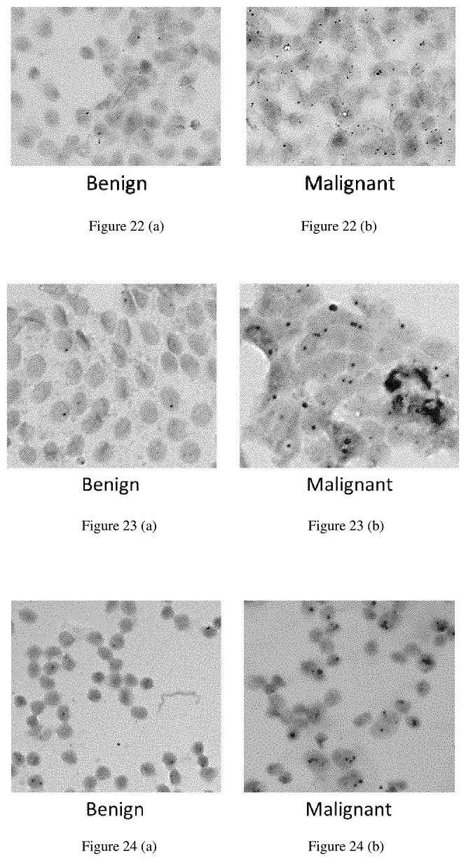

The aspiration biopsy cell samples were obtained by liver puncture. Other methods were the same as in Example 1. The results are shown in FIG. 22(a)-FIG. 22(b).

As shown in FIG. 22(a)-FIG. 22(b), FIG. 22(a) shows a benign liver tumor, and FIG. 22(b) shows a liver cancer. The proportion of cells carrying imprinted genes with loss of imprinting and copy number variation increased gradually with the malignancy of cancer.

### EXAMPLE 11

### Imprinted Gene Analysis for Prostate Aspiration Biopsy Cells

The aspiration biopsy cell samples were obtained by prostate puncture. Other methods were the same as in Example 1. The results are shown in FIG. 23(a)-FIG. 23(b).

As shown in FIG. 23(a)-FIG. 23(b), FIG. 23(a) shows a benign prostate tumor, and FIG. 23(b) shows a prostate cancer. The proportion of cells carrying imprinted genes with loss of imprinting and copy number variation increased gradually with the malignancy of cancer.

### EXAMPLE 12

### Imprinted Gene Analysis for Lymph Node Aspiration Biopsy Cells

The aspiration biopsy cell samples were obtained from lymph nodes adjacent to breast tumor. Other methods were the same as in Example 1. The results are shown in FIG. 24 (a)-FIG. 24 (b).

As shown in FIG. 24(a)-FIG. 24(b), FIG. 24(a) shows aspiration cells from a lymph node adjacent to a benign non-metastatic breast tumor, FIG. 24(b) shows aspiration cells from a lymph node adjacent to a metastatic breast cancer. Only a few cells in the lymph node adjacent to benign breast tumor carried imprinted gene with loss of imprinting, and no cells were found to have imprinted gene with copy number variation, while a large number of cells in the lymph node adjacent to metastatic breast cancer carried imprinted gene with loss of imprinting and imprinted gene with copy number variation.

### EXAMPLE 13

### Imprinted Gene Analysis for Plural Effusion Cells

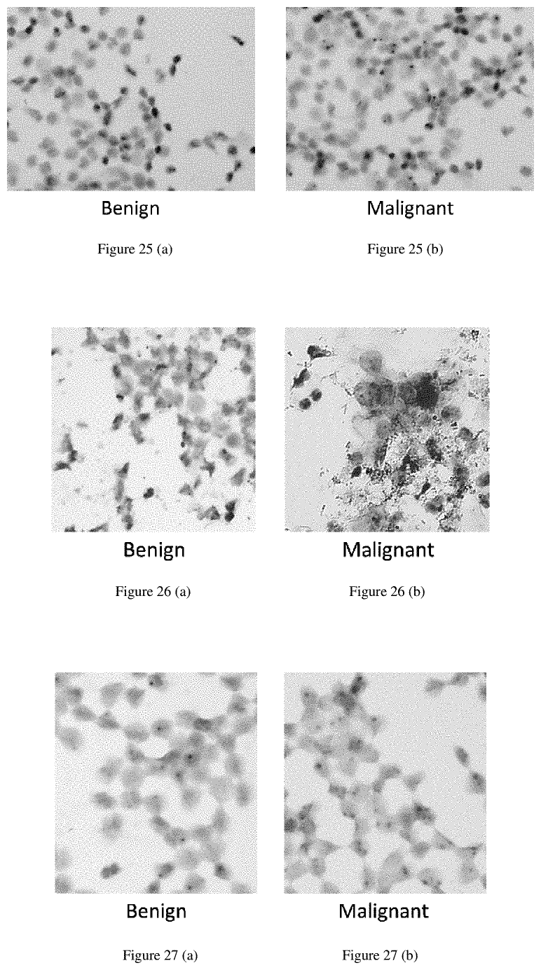

The plural effusion cell samples were obtained by puncture. Other methods were the same as in Example 1. The results are shown in FIG. 25(a)-FIG. 25(b).

As shown in FIG. 25(a)-FIG. 25(b), FIG. 25(a) shows the plural effusion cell from a benign lung tumor patient, and FIG. 25(b) shows the plural effusion cell from a lung cancer patient. Only a few cells in the plural effusion of benign lung tumor patient carried imprinted gene with loss of imprinting, and no cells were found to have imprinted gene with copy number variation, while a large number of cells in the plural effusion of lung cancer patient carried imprinted gene with loss of imprinting and imprinted gene with copy number variation.

### EXAMPLE 14

### Imprinted Gene Analysis for Fecal Exfoliated Cells

The cell samples were obtained from the feces of colorectal tumor patients. Other methods were the same as in Example 1. The results are shown in FIG. 26(a)-FIG. 26(b).

As shown in FIG. 26(a)-FIG. 26(b), FIG. 26(a) shows a benign colorectal tumor, and FIG. 26(b) shows a colorectal cancer. The proportion of cells carrying imprinted genes with loss of imprinting and copy number variation increased gradually with the malignancy of cancer.

### EXAMPLE 15

### Imprinted Gene Analysis for Blood Cell

The cell samples were obtained from peripheral blood of healthy person and patient with leukemia, and the red blood cells were removed using red blood cell lysis solution. Other methods were the same as in Example 1. The results are shown in FIG. 27(a)-FIG. 27(b).

As shown in FIG. 27(a)-FIG. 27(b), FIG. 27(a) shows the blood cell of healthy person and FIG. 27(b) shows the blood cell of patient with leukemia. Only a few cells in the blood sample of healthy person carried imprinted gene with loss of imprinting, and no cells were found to have imprinted gene with copy number variation, while a large number of cells in the blood sample of patient with leukemia carried imprinted gene with loss of imprinting and imprinted gene with copy number variation.

In summary, the method of the present application presents the characteristic of the loss of imprinting on the thyroid sample, breast sample, pancreatic sample, prostate sample, lymph node aspiration cell sample, bronchial brushing cell sample of the lung, urine, sputum, feces, plural effusion cell sample, colonoscopy biopsy sample, and cystoscopy biopsy cell sample in an intuitive manner. By in situ labeling of imprinted genes, objective, intuitive, early, and accurate detection of changes in imprinted genes is achieved, and quantitative model is also provided, making a significant contribution to the early diagnosis of thyroid cancer, breast cancer, pancreatic cancer, lung cancer, liver cancer, colorectal cancer and urinary system cancer.

The Applicant declares that the present application illustrates the detailed methods of the present invention by the above examples, but not limited to the above detailed examples, that is, it does not mean that the present invention must rely on the detailed methods described above to be implemented. It should be apparent to those skilled in the art that any modifications of the present application, the equivalent replacement of each raw material of the products of the present application, the addition of an auxiliary component, the selection of a specific manner, and the like, are all within the scope of protection and disclosure of the present application.

1. A method for cancer diagnosis via biopsy samples, the method comprising:
calculating changes of an expression of imprinted genes with loss of imprinting, an expression of imprinted genes with copy number variation, and a total expression of imprinted genes in a tumor, to grade the expression of the imprinted genes; and
treating the tumor based on a result of grading the expression of the imprinted genes,
wherein the imprinted genes comprise one or a combination of imprinted gene Z1 and imprinted gene Z16, the imprinted gene Z1 is Gnas, and the imprinted gene Z16 is Snrpn/Snurf.

2. The method according to claim 1, wherein:
the imprinted genes further comprise any one or a combination of at least two of Z2, Z3, Z4, Z5, Z6, Z7, Z8, Z9, Z10, Z11, Z12, Z13, Z14 and Z15; wherein the imprinted gene Z2 is Igf2, the imprinted gene Z3 is Peg10, the imprinted gene Z4 is Igf2r, the imprinted gene Z5 is Mest, the imprinted gene Z6 is Plagl1, the imprinted gene Z7 is Cdkn1c, the imprinted gene Z8 is Dcn, the imprinted gene Z9 is Dlk1, the imprinted gene Z10 is Gatm, the imprinted gene Z11 is Grb10, the imprinted gene Z12 is Peg3, the imprinted gene Z13 is Sgce, the imprinted gene Z14 is S1c38a4, the imprinted gene Z15 is Diras3.

3. The method according to claim 2, wherein the expression of an imprinted gene with loss of imprinting and the expression of an imprinted gene with copy number variation are calculated by the following formulas:
total expression of an imprinted gene=(b+c+d)/(a+b+c+d)×100%;
the expression of a normal imprinted gene=b/(b+N+d)×100%;
the expression of an imprinted gene with loss of imprinting=c/(b+c+d)×100%;
the expression of an imprinted gene with copy number variation=d/(b+c+d)×100%;
wherein, “a” represents cell nuclei with no mark inside after performing hematoxylin staining on cells, which means the imprinted gene has no expression in the cell nuclei;
“b” represents cell nuclei with one red/brown mark inside after performing hematoxylin staining on cells, which means the imprinted gene exists in the cell nuclei;
“c” represents cell nuclei with two red/brown marks inside after performing hematoxylin staining on cells, which means the imprinted gene loses imprinting in the cell nuclei; and
“d” represents cell nuclei with more than two red/brown marks inside after performing hematoxylin staining on cells, which means the imprinted gene has copy number variations in the cell nuclei.

4. The method according to claim 3, wherein the expression of an imprinted gene with loss of imprinting, the expression of an imprinted gene with copy number variation, and the total expression of an imprinted gene are classified into 5 grades.

5. The method according to claim 4, wherein the 5 grades are classified respectively according to the expression of an imprinted gene with loss of imprinting, the expression of an imprinted gene with copy number variation, and the total expression of an imprinted gene of imprinted genes Z1 and Z16;
the expression of Z1 and Z16 with loss of imprinting, the expression of Z1 and Z16 with copy number variation, and the total expression of Z1 and Z16 are classified into 5 grades:
grade 0: any one or the combination of at least two of: the expression of Z1 or Z16 with loss of imprinting is less than 15%, the expression of Z1 and Z16 with copy number variation in is less than 2%, or the total expression of Z1 and Z16 is less than 25%;
grade I: any one or the combination of at least two of: the expression of Z1 or Z16 with loss of imprinting is 15-20%, the expression of Z1 and Z16 with copy number variation in is 2-4%, or the total expression of Z1 and Z16 is 25-30%;
grade II: any one or the combination of at least two of: the expression of Z1 or Z16 with loss of imprinting is 20-25%, the expression of Z1 and Z16 with copy number variation in is 4-8%, or the total expression of Z1 and Z16 is 30-40%;
grade III: any one or the combination of at least two of: the expression of Z1 or Z16 with loss of imprinting is 25-35%, the expression of Z1 and Z16 with copy number variation in is 8-12%, or the total expression of Z1 and Z16 is 40-50%;
grade IV: any one or the combination of at least two of: the expression of Z1 or Z16 with loss of imprinting is more than 35%, the expression of Z1 and Z16 with copy number variation in is more than 12%, or the total expression of Z1 and Z16 is more than 50%.

6. The method according to claim 4, wherein the tumor comprises any one or the combination of at least two of thyroid tumor, breast tumor, pancreatic tumor, lung tumor, liver tumor, colorectal tumor, bladder tumor, prostate tumor, gastric tumor, esophagus tumor, nasopharyngeal tumor, oral tumor, ovarian tumor, endometrial tumor, cervical tumor, urinary system tumor, central nervous system tumor, parotid tumor, lymphoma, and leukemia.

7. The method according to claim 6, wherein for thyroid tumor, the expression of Z1 with loss of imprinting, the expression of Z1 with copy number variation, and the total expression of Z1 are classified into 5 grades:
grade 0: any one or the combination of at least two of: the expression of Z1 with loss of imprinting is less than 15%, the expression of Z1 with copy number variation is less than 1.5%, or the total expression of Z1 is less than 40%;
grade I: any one or the combination of at least two of: the expression of Z1 with loss of imprinting is 15-20%, the expression of Z1 with copy number variation is 1.5-4%, or the total expression of Z1 is 40-45%;
grade II: any one or the combination of at least two of: the expression of Z1 with loss of imprinting is 20-30%, the expression of Z1 with copy number variation is 4-8%, or the total expression of Z1 is 45-60%;
grade III: any one or the combination of at least two of: the expression of Z1 with loss of imprinting is 30-40%, the expression of Z1 with copy number variation is 8-15%, or the total expression of Z1 is 60-65%;
grade IV: any one or the combination of at least two of: the expression of Z1 with loss of imprinting is more than 40%, the expression of Z1 with copy number variation is more than 15%, or the total expression of Z1 is more than 65%;
for thyroid tumor, the expression of Z16 with loss of imprinting, the expression of Z16 with copy number variation, and the total expression of Z16 are classified into 5 grades:
grade 0: any one or the combination of at least two of: the expression of Z16 with loss of imprinting is less than 15%, the expression of Z16 with copy number variation is less than 1.5%, or the total expression of Z16 is less than 30%;
grade I: any one or the combination of at least two of: the expression of Z16 with loss of imprinting is 15-20%, the expression of Z16 with copy number variation is 1.5-4%, or the total expression of Z16 is 30-35%;
grade II: any one or the combination of at least two of: the expression of Z16 with loss of imprinting is 20-30%, the expression of Z16 with copy number variation is 4-8%, or the total expression of Z16 is 35-50%;
grade III: any one or the combination of at least two of: the expression of Z16 with loss of imprinting is 30-40%, the expression of Z16 with copy number variation is 8-15%, or the total expression of Z16 is 50-55%;
grade IV: any one or the combination of at least two of: the expression of Z16 with loss of imprinting is more than 40%, the expression of Z16 with copy number variation is more than 15%, or the total expression of Z16 is more than 55%.

8. The method according to claim 6, wherein for breast tumor, the expression of Z1 and Z16 with loss of imprinting, the expression of Z1 and Z16 with copy number variation, and the total expression of Z1 and Z16 are classified into 5 grades:
grade 0: any one or the combination of at least two of: the expression of Z1 and Z16 with loss of imprinting is less than 15%, the expression of Z1 and Z16 with copy number variation is less than 1%, or the total expression of Z1 and Z16 is less than 25%;
grade I: any one or the combination of at least two of: the expression of Z1 and Z16 with loss of imprinting is 15-20%, the expression of Z1 and Z16 with copy number variation is 1-3%, or the total expression of Z1 and Z16 is 25-30%;
grade II: any one or the combination of at least two of: the expression of Z1 and Z16 with loss of imprinting is 20-25%, the expression of Z1 and Z16 with copy number variation is 3-7%, or the total expression of Z1 and Z16 is 30-40%;
grade III: any one or the combination of at least two of: the expression of Z1 and Z16 with loss of imprinting is 25-30%, the expression of Z1 and Z16 with copy number variation is 7-10%, or the total expression of Z1 and Z16 is 40-50%;
grade IV: any one or the combination of at least two of: the expression of Z1 and Z16 with loss of imprinting is more than 30%, the expression of Z1 and Z16 with copy number variation is more than 10%, or the total expression of Z1 and Z16 is more than 50%.

9. The method according to claim 6, wherein for pancreatic tumor, the expression of Z1 and Z16 with loss of imprinting, the expression of Z1 and Z16 with copy number variation, and the total expression of Z1 and Z16 are classified into 5 grades:
grade 0: any one or the combination of at least two of: the expression of Z1 and Z16 with loss of imprinting is less than 15%, the expression of Z1 and Z16 with copy number variation is less than 2%, or the total expression of Z1 and Z16 is less than 20%;
grade I: any one or the combination of at least two of: the expression of Z1 and Z16 with loss of imprinting is 15-20%, the expression of Z1 and Z16 with copy number variation is 2-4%, or the total expression of Z1 and Z16 is 20-30%;
grade II: any one or the combination of at least two of: the expression of Z1 and Z16 with loss of imprinting is 20-25%, the expression of Z1 and Z16 with copy number variation is 4-8%, or the total expression of Z1 and Z16 is 30-40%;
grade III: any one or the combination of at least two of: the expression of Z1 and Z16 with loss of imprinting is 25-30%, the expression of Z1 and Z16 with copy number variation is 8-12%, or the total expression of Z1 and Z16 is 40-50%;
grade IV: any one or the combination of at least two of: the expression of Z1 and Z16 with loss of imprinting is more than 30%, the expression of Z1 and Z16 with copy number variation is more than 12%, or the total expression of Z1 and Z16 is more than 50%.

10. The method according to claim 6, wherein for lung tumor, the expression of Z1 with loss of imprinting, the expression of Z1 with copy number variation, and the total expression of Z1 are classified into 5 grades:
grade 0: any one or the combination of at least two of: the expression of Z1 with loss of imprinting is less than 15%, the expression of Z1 with copy number variation is less than 2%, or the total expression of Z1 is less than 30%;
grade I: any one or the combination of at least two of: the expression of Z1 with loss of imprinting is 15-20%, the expression of Z1 with copy number variation is 2-4%, or the total expression of Z1 is 30-40%;
grade II: any one or the combination of at least two of: the expression of Z1 with loss of imprinting is 20-25%, the expression of Z1 with copy number variation is 4-8%, or the total expression of Z1 is 40-50%;
grade III: any one or the combination of at least two of: the expression of Z1 with loss of imprinting is 25-30%, the expression of Z1 with copy number variation is 8-12%, or the total expression of Z1 is 50-60%;
grade IV: any one or the combination of at least two of: the expression of Z1 with loss of imprinting is more than 30%, the expression of Z1 with copy number variation is more than 12%, or the total expression of Z1 is more than 60%;
for lung tumor, the expression of Z16 with loss of imprinting, the expression of Z16 with copy number variation, and the total expression of Z16 are classified into 5 grades:
grade 0: any one or the combination of at least two of: the expression of Z16 with loss of imprinting is less than 10%, the expression of Z16 with copy number variation is less than 1%, or the total expression of Z16 is less than 25%;
grade I: any one or the combination of at least two of: the expression of Z16 with loss of imprinting is 10-15%, the expression of Z16 with copy number variation is 1-2%, or the total expression of Z16 is 25-30%;
grade II: any one or the combination of at least two of: the expression of Z16 with loss of imprinting is 15-20%, the expression of Z16 with copy number variation is 2-5%, or the total expression of Z16 is 30-40%;
grade III: any one or the combination of at least two of: the expression of Z16 with loss of imprinting is 20-25%, the expression of Z16 with copy number variation is 5-8%, or the total expression of Z16 is 40-50%;
grade IV: any one or the combination of at least two of: the expression of Z16 with loss of imprinting is more than 25%, the expression of Z16 with copy number variation is more than 8%, or the total expression of Z16 is more than 50%.

11. The method according to claim 6, wherein for urinary system tumor, the expression of Z1 and Z16 with loss of imprinting, the expression of Z1 and Z16 with copy number variation, and the total expression of Z1 and Z16 are classified into 5 grades:
grade 0: any one or the combination of at least two of: the expression of Z1 and Z16 with loss of imprinting is less than 17%, the expression of Z1 and Z16 with copy number variation is less than 2%;
grade I: any one or the combination of at least two of: the expression of Z1 and Z16 with loss of imprinting is 17-20%, the expression of Z1 and Z16 with copy number variation is 2-3%;
grade II: any one or the combination of at least two of: the expression of Z1 and Z16 with loss of imprinting is 20-25%, the expression of Z1 and Z16 with copy number variation is 3-7%;
grade III: any one or the combination of at least two of: the expression of Z1 and Z16 with loss of imprinting is 25-30%, the expression of Z1 and Z16 with copy number variation is 7-12%;
grade IV: any one or the combination of at least two of: the expression of Z1 and Z16 with loss of imprinting is more than 30%, the expression of Z1 and Z16 with copy number variation is more than 12%.

12. The method according to claim 6, further comprising:
(1) obtaining a test sample;
(2) designing a probe specific for the sequence of the imprinted genes;
(3) performing in situ hybridization using the probe of step (2) to the test sample; and
(4) analyzing microscopic images and determining the expression status of the imprinted genes;
wherein, the analysis is performed by calculating the expressions of imprinted genes with loss of imprinting, the expressions of imprinted genes with copy number variation, and the total expressions of imprinted genes, and grading the expressions of imprinted genes with loss of imprinting, the expressions of imprinted genes with copy number variation, and the total expressions of imprinted genes to determine the benignity and malignancy of a tumor.

13. The method according to claim 12, wherein the test sample of step (1) is human tissues and/or cells.

14. The method according to claim 13, wherein the test sample comprises any one or the combination of at least two of aspiration biopsy cells, biopsy cells, exfoliated cells, blood sample, or brush biopsy sample.

15. The method according to claim 14, wherein the aspiration biopsy cells comprise anyone or the combination of at least two of the fine or core needle aspiration biopsy samples from thyroid, mammary gland, pancreas, lung, liver, prostate, ovary, lymph node, and parotid;
the biopsy cells comprise biopsy cells from anyone or the combination of at least two of gastroscopy, colonoscopy, cystoscopy, hysteroscopy, or nasopharyngolarygnoscopy;
the exfoliated cells comprise exfoliated cells from anyone or the combination of at least two of urine, sputum, feces, plural effusion, or ascites; and
the brush biopsy samples comprise the brushing samples from anyone or the combination of at least two of bronchus, esophagus, oral cavity, or cervical.

16. The method according to claim 13, wherein the in situ hybridization is performed using the RNAscope in situ hybridization method; and
the RNAscope in situ hybridization is performed by using singleplex or multiplex color assay kit or singleplex or multiplex fluorescence assay kit.

17. The method according to claim 13, wherein the benignity or malignancy of the tumor to be determined is classified as benign tumor, cancer potential, early-stage cancer, medium-stage cancer, and late-stage cancer;
the tumor is determined as a benign tumor, if the expression of both Z1 and Z16 with loss of imprinting are lower than grade I; if the expression of only one of Z1 and Z16 with loss of imprinting is grade I; if the expression of only one of Z1 and Z16 with copy number variation is grade I;
the tumor is determined as cancer potential, if the expression of both Z1 and Z16 with loss of imprinting are grade I; if the expression of both Z1 and Z16 with copy number variation are grade I; if the expression of only one of Z1 and Z16 with loss of imprinting is grade I and the expression of only one of Z1 and Z16 with copy number variation is grade I; if the expression of only one of Z1 and Z16 with loss of imprinting is grade II; if the expression of only one of Z1 and Z16 with copy number variation is grade II;
the tumor is determined as an early-stage cancer, if the expression of both Z1 and Z16 with loss of imprinting are grade II, and/or the expression of both Z1 and Z16 with copy number variation are grade II; if the expression of only one of Z1 and Z16 with loss of imprinting is grade II and the expression of only one of Z1 and Z16 with copy number variation is grade II; if the expression of only one of Z1 and Z16 with loss of imprinting is grade III; if the expression of only one of Z1 and Z16 with copy number variation is grade III;
the tumor is determined as a medium-stage cancer, if the expression of both Z1 and Z16 with loss of imprinting are grade III; if the expression of both Z1 and Z16 with copy number variation are grade III; if the expression of only one of Z1 and Z16 with loss of imprinting is grade III and the expression of only one of Z1 and Z16 with copy number variation is grade III; if the expression of only one of Z1 or Z16 with loss of imprinting is grade IV; if the expression of only one of Z1 or Z16 with copy number variation is grade IV; and
the tumor is determined as a late-stage cancer, if the expression of both Z1 and Z16 with loss of imprinting are grade IV, and/or the expression of both Z1 and Z16 with copy number variation are grade IV.
